(19) United States
(12) Patent Application Publication
(10) Pub. No.: US 2026/0286353 A1
(43) Pub. Date: Sep. 24, 2026

(54) Antisense RNA (asRNA) Technology and Use Thereof

(71) Applicant: 1Globe Health Institute LLC, Norwood, MA (US)

(72) Inventors: Chiang J. LI, Cambridge, MA (US); Xiangao SUN, Norwood, MA (US); Charles LI, Boston, MA (US)

(21) Appl. No.: 19/136,842

(22) PCT Filed: Dec. 8, 2023
(86) PCT No.: PCT/US2023/083252

§ 371 Date: Jun. 6, 2025

Related U.S. Application Data
(60) Provisional application No. 63/431,309, filed on Dec. 8, 2022.

(51) Int. Cl. C12N 15/113

(52) U.S. Cl. CPC C12N 15/113; C12N 2310/11; C12N 2310/311; C12N 2310/3125; C12N 2310/314; C12N 2310/315; C12N 2310/321; C12N 2310/322; C12N 2310/3231; C12N 2310/333; C12N 2310/334; C12N 2310/3341; C12N 2310/335; C12N 2310/336; C12N 2310/351; C12N 2310/3513; C12N 2310/3515

(57) ABSTRACT

The present invention discloses a novel type of gene modulation technology for modulating target nucleic acid and/or protein levels in cells, tissues, organisms and animals. The new technology provides compositions for use in gene modulation applications, including prevention and treatment of human diseases. The composition comprises a short antisense RNA (asRNA) molecule having at least one interspersed motif of deoxyribonucleotide monomer(s). The present invention further provides methods of using the compositions for modulating expression or function of a target gene, or for treatment or prevention of diseases as well as for biomedical research, disease diagnosis and other biological applications.

### CROSS-REFERENCE TO RELATED APPLICATIONS

This application is a national phase application under 35 U.S.C. 371 of international application PCT/US2023/083252, filed Dec. 8, 2023, claims priority to and the benefit of co-pending U.S. provisional patent application Ser. No. 63/431,309, filed Dec. 8, 2022, which application is incorporated herein by reference in its entirety.

### REFERENCE TO SEQUENCE LISTING

Sequence listings and related materials in the XML file named “SEQ-021E1_2026-0605-updated.xml” and created on Jun. 5, 2026, with a size of about 372,924 bytes, is hereby incorporated by reference.

### FIELD OF THE INVENTION

The invention relates to a novel design of short single-stranded antisense RNA oligonucleotides to be used as gene modulation technology as well as related compositions and methods that can be used in the biological or medical research, in the treatment and prevention of diseases and for gene silencing applications in other biological fields.

### BACKGROUND OF THE INVENTION

Single stranded antisense RNAs (asRNA) can be found to occur naturally in both prokaryotic and eukaryotic organisms as a non-coding, typically 19-23 nucleotide-long RNA transcript that is transcribed from the lagging strand of a gene, and is complementary and anti-sense to an mRNA transcript (see Xu, J. et al., 2018). Despite its potential regulatory roles in complex biological networks, asRNA is not well characterized yet—the underlying mechanisms of its functions are still not well understood, and to date only a few in vivo functions of asRNAs have been shown in known organisms (Xu et al., supra). New breakthroughs are needed to unlock the regulatory potential in the antisense activities first observed over 30 years ago in noncoding RNAs, both as a research tool and as a therapeutic agent.

The references cited herein are not admitted to be prior art to the claimed invention.

### SUMMARY OF THE INVENTION

The present invention is based on a surprising discovery that limited gene silencing potency of naturally occurring type of single-stranded antisense RNA comprising exclusively RNAs can be enhanced by introducing interspersed segment of deoxyribonucleotide(s) (“ISD”). Accordingly, the present invention provides a novel type of gene modulation technology enabled by a single-stranded short antisense RNA (asRNA) with at least one ISD.

This novel asRNA with one or more ISD(s) is a short, single-stranded molecule made up by linked nucleotide monomers that are each selected from the group of naturally occurring nucleotide, analogs thereof, and modified nucleotide (hereinafter, collectively referred to as “nucleotide monomers”).

The asRNA molecule of the invention includes “ribonucleotide monomers” selected from the group of naturally occurring ribonucleotide, analogs thereof, and modified ribonucleotide. Further, the gene silencing function of asRNA can be dramatically enabled or enhanced by incorporating one or a few interspersed deoxyribonucleotide monomers. The “deoxyribonucleotide monomers” can be selected from the group of naturally occurring deoxyribonucleotides, analogs thereof, and modified deoxyribonucleotides.

The potent gene silencing effect of the asRNA-based novel platform technology contained in the present disclosure is, in one embodiment, achieved through an antisense oligoribonucleotide that is substantially complementary to a targeted RNA sequence. Our data have shown that asRNA molecules of the present invention, with their unique and novel compositions, can trigger potent gene silencing which are more potent than existing gene silencing technologies, and therefore enabling reduction of dose-dependent toxicities. The asRNA molecules of the present invention are also expected to have at least one of the following advantages over existing gene silencing technologies including better tissue penetration; enabling gene silencing in cytoplasm as well as in nuclei/nucleus and mitochondria/mitochondrion; reduced off-target effects; better stability; lower synthesis cost and other improved pharmaceutical properties. Therefore, the asRNA molecules of the present invention have great potential for addressing a variety of challenges facing existing gene silencing technologies. The asRNA molecules of the present invention can be used in all areas that current gene silencing oligonucleotides are being applied or contemplated for use, including research, diagnosis, disease prevention and therapies as well as other applications in biological fields, including agriculture and veterinary medicine.

In a first aspect, the present invention provides a composition comprising a short antisense RNA (asRNA) molecule having a single strand of linked ribonucleotide monomers, where the strand is substantially complementary to a targeted segment of a targeted RNA through at least one targeting region. Further, the asRNA molecule includes at least one interspersed segment of deoxyribonucleotide monomer(s) (ISD) that has at least one deoxyribonucleotide monomer. The ribonucleotide monomer in the molecule is selected from the group consisting of a naturally occurring ribonucleotides, an analog thereof, and a modified ribonucleotide; and the interspersed segment of deoxyribonucleotide monomer in the asRNA molecule is selected from the group consisting of a naturally occurring deoxyribonucleotide, an analog thereof, and a modified deoxyribonucleotide. In an embodiment, the ISD in the asRNA molecule has at least 1, 2, 3, 4, 5, 6, 7, 8, 9 or 10 deoxyribonucleotide monomer(s). In a feature, the ISD in the asRNA molecule has at least 2, 3, 4, 5, 6, 7 or 8 contiguous deoxyribonucleotide monomers. In an embodiment, the ISD includes at least 2 deoxyribonucleotide monomers. There may be more than one ISD in the asRNA molecule. In a feature, each ISD, independently of each other, either consists of one deoxyribonucleotide monomer, or comprises at least 2, 3, 4, 5 or more contiguous deoxyribonucleotide monomers. In one feature, the ISD is disposed in at least one targeting region. In some embodiments, ISD can be deposited at any position of the asRNA molecule. In some embodiments, ISD(s) is/are positioned at a more central part (at least 1, 2, 3, 4, 5, 6, 7 or 8 nucleotide(s) away from both ends, i.e., starting from position no. 2 or more central counting from both ends) of the asRNA molecule. In some embodiments, ISD(s) comprise at least one deoxyribonucleotide monomer positioned at the 5′ end and/or the 3′ end of the asRNA molecule.

In a feature, at least one gene modulation property or pharmaceutical property is better or more desirable when a single-stranded antisense RNA includes at least one ISD; the property is selected from the group of: efficacy, potency, speed of onset, durability, synthesis economy, off-target effects, non-specific immune stimulation, stability, and delivery. More specifically, improved gene modulation properties or pharmaceutical properties of the asRNA molecule of the present invention, when compared to a corresponding single-stranded antisense RNA without ISD, means, for example, one or more of the following is true: better efficacy and/or potency, quicker onset of action, improved pharmacokinetic properties, longer durability, reduced off-target effects, less dosage-dependent stereotypic toxicity, avoidance of non-specific interferon-like response, and lower manufacture cost, better stability, and better delivery.

The composition provided by the present invention is used for modulating gene expression or function in a eukaryotic cell, wherein the asRNA with ISD (asRNA-ISD) is caused to contact a cell or administered to a subject.

In one feature, the asRNA molecule includes multiple linked nucleotide monomers forming a nucleobase sequence, and is at least 70%, 80%, 85%, 90%, 95% complementary or fully complementary to the targeted segment of the targeted RNA. In certain embodiments, the targeted RNA is either mRNA, pre-mRNA, mt-mRNA and or non-coding RNA where the RNA either encodes a protein or regulates a part of a biological pathway implicated in a disease, e.g., a mammalian disease. The terms “target” and “targeted” are used interchangeably in the present disclosure and share the same meaning.

In various embodiments, the asRNA molecule has a backbone length of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 linked nucleotide monomers, or equivalents thereof, or of a range bracketed by any two of the above values (both range endpoints included). For example, some of the ranges of the length of the asRNA include: 8-48 nucleotide monomers; 8-44 nucleotide monomers; 8-42 nucleotide monomers; 8-40 nucleotide monomers; 8-36 nucleotide monomers; 8-33 nucleotide monomers; 10-48 nucleotide monomers; 10-44 nucleotide monomers; 10-42 nucleotide monomers; 10-40 nucleotide monomers; 10-36 nucleotide monomers; 10-30 nucleotide monomers; 10-29 nucleotide monomers; 12-48 nucleotide monomers; 12-44 nucleotide monomers; 12-42 nucleotide monomers; 12-40 nucleotide monomers; 12-36 nucleotide monomers; 12-34 nucleotide monomers; 12-32 nucleotide monomers; 12-30 nucleotide monomers; 12-29 nucleotide monomers; 12-28 nucleotide monomers; 12-26 nucleotide monomers; 12-25 nucleotide monomers; 13-48 nucleotide monomers; 13-44 nucleotide monomers; 13-42 nucleotide monomers; 13-40 nucleotide monomers; 13-36 nucleotide monomers; 13-34 nucleotide monomers; 13-32 nucleotide monomers; 13-30 nucleotide monomers; 13-28 nucleotide monomers; 13-26 nucleotide monomers; 13-25 nucleotide monomers; 13-24 nucleotide monomers; 13-23 nucleotide monomers; 14-36 nucleotide monomers; 15-23 nucleotide monomers; 20-36 nucleotide monomers; 21-36 nucleotide monomers; 24-36 nucleotide monomers; at least 21 nucleotide monomers; at least 24 nucleotide monomers and at least 8 nucleotide monomers.

In a feature of the asRNA molecule of the invention, at least one nucleotide monomer in the strand is a modified nucleotide or nucleotide analogue, e.g., a sugar-, backbone-, and/or base-modified nucleotide. In an embodiment, such a backbone-modified nucleotide has at least a modification in an internucleoside linkage, e.g., to include at least one of a nitrogen or sulphur heteroatom. In some embodiments, the modified internucleoside linkage is or includes: phosphorothioate (P═S) group, phosphotriesters, methylphosphonates, or phosphoramidate.

In certain embodiments, the asRNA molecule includes at least one modified internucleoside linkage that is a phosphorothioate internucleoside linkage. In some embodiments, each internucleoside linkage of the asRNA molecule is a phosphorothioate internucleoside linkage. In various embodiments, the internucleoside linkages are a mixture of phosphorothioate and phosphodiester linkages.

In a feature, the asRNA molecule of the invention has at least one modified nucleotide or nucleotide analogue that includes a modified sugar moiety. In certain embodiments, the 2′ position of the modified sugar moiety is replaced by a group selected from OR, R, halo, SH, SR, NH₂, NHR, NR₂, or CN, where each R is independently C₁-C₆ alkyl, alkenyl or alkynyl, and halo is F, Cl, Br or I. In some embodiments, the 2′ position of the modified sugar moiety is replaced by a group selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), or O—CH₂—C(═O)—N(R₁)—(CH₂)₂—N(Rₘ)(Rₙ), where each Rₗ, Rₘ and Rₙ is, independently, H or substituted or unsubstituted C₁-C₁₀ alkyl.

In some embodiments, the modified sugar moiety has substituent group(s) selected from the group of 5′-vinyl, 5′-methyl (R or S), 4′-S, 2′-F, 2′-OCH₃, 2′-OCH₂CH₃, 2′-OCH₂CH₂F, 2′-O-aminopropylation (2′-AP) and 2′-O(CH₂)₂OCH₃. In some embodiments, the modified sugar moiety is substituted by a bicyclic sugar selected from the group of 4′-(CH₂)—O-2′ (LNA); 4′-(CH₂)—S-2; 4′-(CH₂)₂—O-2′ (ENA); 4′-CH(CH₃)—O-2′ (cEt) and 4′-CH(CH₂OCH₃)—O-2′, 4′-C(CH₃)(CH₃)—O-2′, 4′-CH₂—N(OCH₃)-2′, 4′-CH₂—O—N(CH₃)-2′, 4′-CH₂—N(R)—O-2′(where R is H, C₁-C₁₂ alkyl, or a protecting group), 4′-CH₂—C(H) (CH₃)-2′, and 4′-CH₂—C—(═CH₂)-2′. In some embodiments, the modified sugar moiety is selected from the group of 2′-O-methoxyethyl modified sugar (MOE), a 4′-(CH₂)—O-2′ bicyclic sugar (LNA), 2′-deoxy-2′-fluoroarabinose (a 2′-F-arabino, FANA), and a methyl(methyleneoxy) (4′-CH(CH₃)—O-2) bicyclic sugar (cEt).

In a feature of the asRNA molecule of the invention, the sugar moiety of the deoxyribonucleotide monomer is either the sugar moiety of a naturally occurring deoxyribonucleotide (2-H) or 2′-deoxy-2′-fluoroarabinose (FANA).

In a feature of the asRNA molecule of the invention, the sugar moiety of the ribonucleotide monomer is selected from the group of a naturally occurring ribonucleotide (2-OH), 2′-F modified sugar, 2′-OMe modified sugar, 2′-O-methoxyethyl modified sugar (MOE), a 4′-(CH₂)—O-2′ bicyclic sugar (LNA) and a methyl(methyleneoxy) (4′-CH(CH₃)—O-2) bicyclic sugar (cEt).

In another feature, the asRNA molecule of the invention includes at least one nucleotide monomer having a modified nucleobase. In some embodiments, the modified nucleobase is selected from the group of: 5-methylcytosine (5-Me-C), inosine base, a tritylated base, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 1-methyl-pseudo-uracil, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 1-methyl-pseudo-uracil, 8-halo, 8-amino, 8-thiol, 8-thioalkyl, 8-hydroxyl and other 8-substituted adenines and guanines, 5-halo particularly 5-bromo, 5-trifluoromethyl, 5-methyl uridine and other 5-substituted uracils and cytosines, 7-methylguanine and 7-methyladenine, 2-F-adenine, 2-aminoadenine, 8-azaguanine and 8-azaadenine, and 7-deazaguanine and 7-deazaadenine and 3-deazaguanine and 3-deazaadenine. In a particular embodiment, the modified nucleobase is a 5-methylcytosine. In an embodiment, each cytosine base in the molecule of the invention is 5-methylcytosine.

In a feature of the invention, the asRNA molecule is used for modulating gene expression or function in a cell, e.g., a eukaryotic cell such as a mammalian cell.

In certain embodiments, the RNA targeted by the asRNA molecule of the invention is selected from mRNA, pre-mRNA, mt-mRNA and non-coding RNA. In one feature, such targeted RNA either encodes a protein or regulates a part of a biological pathway implicated in a disease. Such target RNA, in various embodiments, can be, but are not limited to, selected from: an mRNA, a pre-mRNA, a mt-mRNA, a non-coding RNA or a lncRNA of a gene implicated in human or animal diseases or condition; an mRNA or a pre-mRNA of a gene of a pathogenic microorganism; a viral RNA, and an RNA implicated in a disease selected from the group consisting of autoimmune diseases, inflammatory diseases, degenerative diseases, infectious diseases, proliferative diseases, metabolic diseases, immune-mediated disorders, allergic diseases, dermatological diseases, malignant diseases, gastrointestinal disorders, respiratory disorders, cardiovascular disorders, renal disorders, rheumatoid disorders, neurological disorders, endocrine disorders, and aging-related disorders or diseases.

In one feature, the asRNA molecule of the invention is conjugated to a ligand or a moiety. In certain embodiments, the ligand or moiety is selected from the group of: peptide/protein, antibody, polymer, polysaccharide, lipid, hydrophobic moiety or molecule, cationic moiety or molecule, lipophilic compound or moiety oligonucleotide, cholesterol, GalNAc and aptamer.

In a second aspect, the present invention provides a pharmaceutical composition comprising the composition in the first aspect as active agent, and a pharmaceutically acceptable excipient, carrier, or diluent. Examples of such carriers include and are not limited to: a pharmaceutical carrier, a positive-charge carrier, a lipid nanoparticle, a liposome, a protein carrier, a hydrophobic moiety or molecule, a cationic moiety or molecule, GalNAc, a polysaccharide a polymer, a nanoparticle, a nanoemulsion, a cholesterol, a lipid, a lipophilic compound or moiety, and a lipoid.

In a third aspect, the present invention provides a method of using the composition in the first aspect or the pharmaceutical composition in the second aspect for treating or preventing a disease or a condition by administering a therapeutically effective amount of an asRNA molecule of the invention or a pharmaceutical composition containing such a molecule to a subject in need thereof. The administration method is a route selected from the group of intravenous injection (iv), subcutaneous injection (sc), per os (po), intramuscular (im) injection, oral administration, inhalation, topical, intrathecal, and other regional administrations.

In a feature, the disease or condition being prophylactically or therapeutically treated is selected from the group of cancer, autoimmune disease, inflammatory diseases, degenerative diseases, infectious diseases, proliferative diseases, metabolic diseases, immune-mediated disorders, allergic diseases, dermatological diseases, malignant diseases, gastrointestinal disorders, hepatic disorders, respiratory disorders, cardiovascular disorders, dermatological disorders, renal disorders, rheumatoid disorders, neurological disorders, psychiatric disorders, endocrine disorders, and aging-related disorders or diseases.

In a fourth aspect, the present invention provides a method of using the composition in the first aspect or the pharmaceutical composition in the second aspect for regulating or modulating a gene expression or gene function in a eukaryotic cell. The method comprises the step of contacting the cell with an effective amount of any asRNA molecule of the invention or a pharmaceutical composition containing such a molecule.

In one embodiment, said contacting step comprises the step of introducing a composition containing said asRNA molecule into a target cell in culture or in an organism in which the selective gene silencing can occur. In a further embodiment, the introducing step is selected from the group consisting of simple mixing, transfection, lipofection, electroporation, infection, injection, oral administration, intravenous injection (iv), subcutaneous injection (sc), per os (po), intramuscular (im) injection, inhalation, topical, intrathecal, and other regional administrations. In another embodiment, the introducing step comprises using a pharmaceutically acceptable excipient, carrier, or diluent selected from the group that includes a pharmaceutical carrier, a positive-charge carrier, a lipid nanoparticle, a liposome, a protein carrier, a hydrophobic moiety or molecule, a cationic moiety or molecule, GalNAc, a polysaccharide a polymer, a nanoparticle, a nanoemulsion, a cholesterol, a lipid, a lipophilic compound or moiety, and a lipoid.

In certain embodiments, the target gene is mRNA. In certain embodiments, the target gene is pre-mRNA. In certain embodiments, the target gene is mt-mRNA. In certain embodiments, the target gene is non-coding RNA, such as microRNA and lncRNA.

In an embodiment, the target gene is associated with a disease, a pathological condition, or an undesirable condition in a mammal. In a further embodiment, the target gene is a gene of a pathogenic microorganism. In an even further embodiment, the target gene is a viral gene. In another embodiment, the target gene is a tumor-associated gene. In yet another embodiment, the target gene is a gene associated with a disease selected from the group listed with respect to the third aspect.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention. While several embodiments have been shown and described, any modifications may be made without departing from the spirit and scope of the present invention.

### BRIEF DESCRIPTION OF FIGURES

FIG. 1 illustrates exemplary structures of some embodiments of asRNAs with various motif of interspersed segment of deoxyribonucleotide monomers (ISD) and corresponding antisense single-stranded RNA without ISD (ASR), and shows exemplary sequences of the asRNAs and ASR having the illustrated exemplary structures for targeting the APOCIII gene.

FIG. 2 shows the gene silencing potency of asRNAs having structures in FIG. 1 targeting the APOCIII gene in comparison with corresponding ASR. Relative mRNA levels of the APOCIII gene were determined after the asRNAs and corresponding ASR at 10 nM were introduced into HepaRG cells via transfection.

FIG. 3 illustrate exemplary structures of some embodiments of asRNAs with various positions of ISD and exemplary sequences of the asRNAs for targeting the APOCIII gene.

FIG. 4 shows the gene silencing potency of asRNAs having sequences in FIG. 3 targeting the APOCIII gene. Relative mRNA levels of the APOCIII gene were determined after the asRNAs at 10 nM were introduced into HepaRG cells via transfection.

FIG. 5A illustrates exemplary sequences of some embodiments of asRNAs with various lengths. FIGS. 5B and 5C show the gene silencing potency for targeting the APOCIII gene of asRNAs shown in FIG. 5A at different concentrations. The gene silencing potency of relative mRNA levels of the APOCIII gene were determined after the asRNAs at 100 μM and 10 nM were introduced into HepaRG cells via transfection.

### DETAILED DESCRIPTION OF THE INVENTION

The present invention refers to gene or RNA modulation/silencing technology using a novel type of short single-stranded antisense RNAs interspersed with DNA monomers. This new technology is used for modulation of gene expression or function in vitro and in vivo by using a short single-stranded antisense RNA with interspersed segment of deoxynucleotides composition. The present invention also provides methods of using the compositions for modulating expression or function of a target gene, or for treatment or prevention of diseases as well as for biomedical research and other biological applications.

### 1. DEFINITIONS

As used herein, the singular form “a”, “an”, and “the” include plural references unless the context clearly dictates otherwise. For example, the term “a cell” includes a plurality of cells including mixtures thereof.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

As used herein, the term “analog” or “analogue,” interchangeably, means a functional or structural equivalent. For instance, nucleoside and nucleotide analogues have been used in clinical treatment of cancer and viral infections for decades and new compounds are continually synthesized and evaluated by the researchers and the pharmaceutical industry, see, e.g., Jordheim L. P. et al., Nat Rev Drug Discov 12, 447-464 (2013).

As used herein, the term “deoxyribonucleoside monomer” means a nucleoside monomer that includes a naturally occurring deoxyribonucleoside, an analog thereof, and a modified deoxyribonucleoside. The term “deoxyribonucleotide monomer” means a nucleotide monomer that includes a naturally occurring deoxyribonucleotide, an analog thereof, and a modified deoxyribonucleotide.

As used herein, the term “ribonucleoside monomer” means a nucleoside monomer that includes a naturally occurring ribonucleoside, an analog thereof, and a modified ribonucleoside. The term “ribonucleotide monomer” means a nucleotide monomer that includes a naturally occurring ribonucleotide, an analog thereof, and a modified ribonucleotide.

As used herein, the term “nucleoside” means a compound comprising a nucleobase moiety and a sugar moiety. Nucleoside monomers include, but are not limited to, naturally occurring nucleosides (e.g., deoxyribonucleosides and ribonucleosides as found in DNA and RNA, respectively), analogs thereof and modified nucleosides. A nucleoside monomer can be either a deoxyribonucleoside monomer or a ribonucleoside monomer. Nucleoside monomers may be linked to a phosphate moiety to become, for example, nucleotide monomers.

As used herein, the term “nucleotide” means a nucleoside further comprising a phosphate linking group. Nucleotide monomers include, but are not limited to, naturally occurring nucleotides (e.g., deoxyribonucleotides and ribonucleotides as found in DNA and RNA, respectively), analogs thereof and modified nucleotides. A nucleotide monomer can be either a deoxyribonucleotide monomer or a ribonucleotide monomer. A modified nucleotide may be modified at one of more of the following: its nitrogen-containing nucleobase moiety, its five-carbon sugar moiety, and its phosphate linking group that results in changes in the internucleoside linkage.

As used herein, the term “oligo” or “oligonucleotide” refers to a compound comprising a plurality of linked nucleoside monomers. In certain embodiments, one or more of nucleoside monomers or one or more of the internucleoside linkages are modified.

The terms “deoxynucleoside” and “deoxyribonucleoside” are used interchangeably herein. The terms “deoxynucleotide” and “deoxyribonucleotide” are also used interchangeably herein. As used herein, a “deoxynucleoside” or “deoxynucleotide” is a nucleoside or nucleotide, respectively, that contains a deoxy sugar moiety.

As used herein, the term “motif” means the pattern of chemically distinct regions, e.g., in an oligonucleotide strand.

As used herein, the term “immediately adjacent” means there are no intervening elements in between two elements, for example, between regions, segments, nucleotides and/or nucleosides.

As used herein, the term “modified nucleotide” means a nucleotide having at least one modified sugar moiety, modified internucleoside linkage, and/or modified nucleobase.

As used herein, the term “modified nucleoside” means a nucleoside having at least one modified sugar moiety, and/or modified nucleobase.

As used herein, the term “modified oligonucleotide” means an oligonucleotide comprising at least one modified nucleotide.

As used herein, the term “naturally occurring internucleoside linkage” means a 3′ to 5′ phosphodiester linkage.

As used herein, the term “modified internucleoside linkage” refers to a substitution or any change from a naturally occurring internucleoside bond. For example, a phosphorothioate linkage is a modified internucleoside linkage.

As used herein, the term “natural sugar moiety” means a sugar naturally found in DNA (2-H) or RNA (2-OH).

As used herein, the term “modified sugar” refers to a substitution or change from a natural sugar. For example, a 2′-O-methoxyethyl modified sugar is a modified sugar.

As used herein, the term “bicyclic sugar” means a furosyl ring modified by the bridging of two non-geminal ring atoms. A bicyclic sugar is a modified sugar.

As used herein, the term “bicyclic nucleic acid,” “BNA,” “bicyclic nucleoside,” or “bicyclic nucleotide” refers to a nucleoside or nucleotide where the furanose portion of the nucleoside or nucleotide includes a bridge connecting two carbon atoms on the furanose ring, thereby forming a bicyclic ring system.

As used herein, the term “2′-O-methoxyethyl” (also 2′-MOE, 2′-O(CH₂)₂—OCH₃ and 2′-O-(2-methoxyethyl)) refers to an O-methoxy-ethyl modification of the 2′ position of a furosyl ring. A 2′-O-methoxyethyl modified sugar is a modified sugar. As used herein, the term “2′-O-methoxyethyl nucleotide” means a modified nucleotide comprising a 2′-O-methoxyethyl modified sugar moiety.

As used herein, the term “modified nucleobase” refers to any nucleobase other than adenine, cytosine, guanine, thymidine, or uracil. For example, 5-methylcytosine is a modified nucleobase. In contrast, an “unmodified nucleobase,” as used herein, means the purine bases adenine (A) and guanine (G), and the pyrimidine bases thymine (T), cytosine (C), and uracil (U).

As used herein, the term “5-methylcytosine” means a cytosine modified with a methyl group attached to the 5′ position. A 5-methylcytosine is a modified nucleobase.

As used herein, “RNA-like nucleotide” means a modified nucleotide that adopts a northern configuration and functions like RNA when incorporated into an oligonucleotide. RNA-like nucleotides include but are not limited to bridged nucleic acid (BNA), LNA, cEt, 2′-O-methylated nucleotide, 2′-O-methoxyethylated (2′-MOE) nucleotide, 2′-fluorinated nucleotide, 2′-O-aminopropylated (2′-AP) nucleotide, tricyclo-DNA (tcDNA) and RNA surrogates.

As used herein, “DNA-like nucleotide” means a modified nucleotide that functions like DNA when incorporated into an oligonucleotide. DNA-like nucleotides include but are not limited to 2′-deoxy-2′-fluoroarabinose (FANA) nucleotides and DNA surrogates.

As used herein, “non-coding RNA” means an RNA molecule that is not translated into a protein. Examples of non-coding RNAs include transfer RNAs (tRNAs) and ribosomal RNAs (TRNAs), as well as small non-coding RNAs and the long ncRNAs (lncRNAs). As used herein, examples of “small non-coding RNA” includes, but are not limited to, microRNAs (miRNAs), asRNA, pre-miRNAs, pri-miRNAs, piRNAs, snoRNAs, snRNAs, exRNAs, scaRNAs and mimics of any of the foregoing. As used herein, “lncRNA”, “long non-coding RNA” are transcribed RNA molecules containing greater than 200 nucleotides that do not code for protein. LncRNAs can also be subjected to common post-transcriptional modifications, including 5′-capping, 3′-polyadenylation, and splicing. Generally, lncRNA are a diverse class of molecules that play a variety of roles in modulation of gene and genome function. For example, lncRNAs are known to regulate gene transcription, translation, and epigenetic regulation. Examples of lncRNAs include, but are not limited to Konqlotl, Xlsirt, Xist, ANRIL, NEAT1, NRON, DANCR, OIP5-AS1, TUG1, CasC7, HOTAIR and MALAT1. As used herein, “splice” or “splicing” refers to a natural process that removes unnecessary regions of RNA and reforms the RNA. An example of modulation of RNA target function by oligonucleotides is modulation of non-coding RNA function. In some embodiments, the asRNA is designed to target one of the foregoing small non-coding RNAs. In some embodiments, the asRNA is designed to target miRNA. In some embodiments, the asRNA is designed to target pre-miRNA. In some embodiments, the asRNA is designed to target pri-miRNA. In some embodiments, the asRNA is designed to target lncRNA. In some embodiments, the asRNA is designed to target splice.

The targeted RNAs in nuclei/nucleus refers to RNA molecules which are synthesized and/or function in the nucleus of a cell. According to preferred embodiments the targeted RNAs in nuclei/nucleus of the present invention include non-coding RNA, lncRNA, pre-mRNA and pre-miRNA. As used herein, the term “pre-mRNA” means an unprocessed or partially processed precursor mRNA containing introns and exons, which is synthesized from the cellular DNA template by transcription. Pre-mRNA requires splicing (removal) of introns to produce the mRNA molecule containing only exons. In some embodiments, the asRNA is designed to target pre-mRNA. The term “mt-mRNA” refers to the mRNA molecules which are transcribed from the mitochondria DNA. In some embodiments, the asRNA is designed to target mt-mRNA in mitochondria.

The term “interspersed” as used herein refers to having a different kind of moiety at an adjacent space, for instance, by a different kind of nucleotide or nucleotide analogue, a different modification on the same kind of nucleotide or nucleotide analogue. In various embodiments of the invention, an “interspersed segment of deoxyribonucleotide monomer(s) (ISD)” refers to a section in an oligonucleotide strand where one or multiple deoxyribonucleotide(s) are connected to at least one moiety that is a different kind from said deoxyribonucleotide(s). For example: if said deoxyribonucleotide(s) are unmodified, then a different kind of moiety may be a ribonucleotide or an analog thereof, a modified ribonucleotide, a modified deoxyribonucleotide, or a deoxyribonucleotide analog. if said deoxyribonucleotide(s) are modified, then a different kind of moiety may be a ribonucleotide or an analogue thereof, a modified ribonucleotide, an unmodified deoxyribonucleotide, a differently modified deoxyribonucleotide, or a different kind of deoxyribonucleotide analog.

As used herein, “modulating”, “regulating” and its grammatical equivalents refer to either increasing or decreasing (e.g., silencing), in other words, either up-regulating or down-regulating. As used herein, “gene silencing” refers to reduction of gene expression and may refer to a reduction of gene expression about 20%, 30%, 40%, 50%, 60%, 70%, 80%, 90% or 95% of the targeted gene.

As used herein, the terms “inhibiting”, “to inhibit” and their grammatical equivalents, when used in the context of a bioactivity, refer to a down-regulation of the bioactivity, which may reduce or eliminate the targeted function, such as the production of a protein or the phosphorylation of a molecule. In particular embodiments, inhibition may refer to a reduction of about 20%, 30%, 40%, 50%, 60%, 70%, 80%, 90% or 95% of the targeted activity. When used in the context of a disorder or disease, the terms refer to success at preventing the onset of symptoms, alleviating symptoms, or eliminating the disease, condition or disorder.

As used herein, the term “substantially complementary” or “complementary” refers to complementarity in a base-paired, double-stranded region between two chains of linked nucleosides and not any single-stranded region such as a terminal overhang. The complementarity does not need to be perfect; there may be any number of base pair mismatches, for example, between the two chains of linked nucleosides. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, substantially complementary sequences can refer to sequences with base-pair complementarity of at least, 70%, 75%, 80%, 85%, 86%, 87%, 88%, 89%, 90%, 91%, 92%, 93%, 94%, 95%, 96%, 97%, 98%, 99%, or 100%, or any number in between, in a double-stranded region.

As used herein, “fully complementary” or “100% complementary” means each nucleobase of a nucleobase sequence of a first strand of linked nucleosides has a complementary nucleobase in a second nucleobase sequence of a second strand of linked nucleosides. In certain embodiments, a first strand of linked nucleosides is a target compound and a second strand of linked nucleosides is an antisense compound or vice versa.

As used herein, the term “targeting region” refers to a region in an oligonucleotide strand that is substantially or fully complementary to another oligonucleotide strand such that the two strands, under the right conditions, hybridize or anneal to each other at such targeting region. For example, an antisense strand can include a targeting region through which it can hybridize with a targeted mRNA.

The terms “administer,” “administering,” or “administration” are used herein in their broadest sense. These terms refer to any method of introducing to a subject a compound or pharmaceutical composition described herein and can include, for example, introducing the compound systemically, locally, or in situ to the subject. Thus, a compound of the present disclosure produced in a subject from a composition (whether or not it includes the compound) is encompassed in these terms. When these terms are used in connection with the term “systemic” or “systemically,” they generally refer to in vivo systemic absorption or accumulation of the compound or composition in the blood stream followed by distribution throughout the entire body.

The terms “effective amount” and “therapeutically effective amount” refer to that amount of a compound or pharmaceutical composition described herein that is sufficient to affect the intended result including, but not limited to, disease treatment, as illustrated below. In some embodiments, the “therapeutically effective amount” is the amount that is effective for detectable killing or inhibition of the growth or spread of cancer cells, the size or number of tumors, and/or other measure of the level, stage, progression and/or severity of the cancer. In some embodiments, the “therapeutically effective amount” refers to the amount that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose may vary depending on, for example, the particular pharmaceutical composition, subject and their age and existing health conditions or risk for health conditions, the dosing regimen to be followed, the severity of the disease, whether it is administered in combination with other agents, timing of administration, the tissue to which it is administered, and the physical delivery system in which it is carried.

The term “cancer” in a subject refers to the presence of cells possessing characteristics typical of cancer-causing cells, such as uncontrolled proliferation, immortality, metastatic potential, rapid growth and proliferation rate, and certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, peritoneal cancer, colon cancer, rectal cancer, colorectal adenocarcinoma, cancer of the anal region, stomach cancer, gastric cancer, gastrointestinal cancer, gastric adenocarcinoma, adrenocorticoid carcinoma, uterine cancer, carcinoma of the fallopian tubes, carcinoma of the endometrium, carcinoma of the vagina, carcinoma of the vulva, Hodgkin's Disease, esophageal cancer, gastroesophageal junction cancer, gastroesophageal adenocarcinoma, chondrosarcoma, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and are included in this term. For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

The term “pharmaceutical composition” is a formulation containing the active ingredient, e.g., the molecule or composition disclosed herein, in a form suitable for administration to a subject, often in mixture with other substances, e.g., a pharmaceutical carrier such as a sterile aqueous solution. In one embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient in a unit dose of composition is an effect the amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, and the like. Dosage forms for the topical or transdermal administration of an asRNA of this invention include powders, sprays, ointments, pastes, creams, lotions, gels, solutions, patches and inhalants.

The term “pharmaceutical agent” means a substance that provides a therapeutic benefit when administered to an individual.

The term “pharmaceutically acceptable carrier” means a medium or diluent that does not interfere with the structure of the compound. Certain of such carriers enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection, infusion or topical administration. For example, a pharmaceutically acceptable carrier can be a sterile aqueous solution.

The term “pharmaceutically acceptable derivative” encompasses derivatives of the compounds described herein such as solvates, hydrates, esters, prodrugs, polymorphs, isomers, isotopically labelled variants, pharmaceutically acceptable salts and other derivatives known in the art.

The term “pharmaceutically acceptable salts” means physiologically and pharmaceutically acceptable salts of compounds, i.e., salts that retain the desired biological activity of the parent compound and do not impart undesired toxicological effects thereto. The term “pharmaceutically acceptable salt” or “salt” includes a salt prepared from reacting the parent compound with pharmaceutically acceptable non-toxic acids or bases, including inorganic or organic acids and bases. Pharmaceutically acceptable salts of the compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). Pharmaceutically acceptable salt can include, but is not limited to, acid addition salts including hydrochlorides, hydrobromides, phosphates, sulphates, hydrogen sulphates, alkylsulphonates, arylsulphonates, acetates, benzoates. citrates, maleales, fumarates, succinates, lactates, and tartrates; alkali metal cations such as Na, K, Li, alkali earth metal salts such as Mg or Ca, or organic amine salts. In particular, sodium salts of oligonucleotides have proven to be useful and are well accepted for therapeutic administration to humans. Accordingly, in one embodiment, the compounds described herein are in the form of a sodium salt.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Terms such as “treating” or “treatment” or “to treat” or “alleviating” or “to alleviate” as used herein refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus, those in need of treatment include those already with the disorder; those prone to have the disorder; and those in whom the disorder is to be prevented. A subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced morbidity and mortality; and improvement in quality of life.

The term “carrier” as used herein means a pharmaceutically acceptable material, composition or vehicle, such as, for example, a liquid or solid filler, diluent, excipient, solvent or encapsulating material involved in or capable of carrying or transporting the subject pharmaceutical compound from one organ, or portion of the body, to another organ, or portion of the body. Each carrier must be “acceptable” in the sense of being compatible with the other ingredients of the formulation and not injurious to the patient. Non-limiting examples of pharmaceutically acceptable carriers, carriers, and/or diluents include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

### 2. CERTAIN EMBODIMENTS

Certain embodiments of the present invention provide an asRNA composition made of linked ribonucleoside monomers with at least one interspersed segment of deoxyribonucleoside monomer(s) called ISD(s). Some or all of the nucleoside monomers contained therein and/or the internucleoside linkage(s) may be modified from those found in natural RNAs or DNAs. One or more ISDs may be found in the asRNA of the invention. In some embodiments, each ISD independently consists of 1 deoxyribonucleotide monomer or at least 2, 3, 4, 5, 6, 7, 8, 9, or 10 contiguous deoxyribonucleotide monomers. In some embodiments, an ISD has at least two contiguous and linked deoxyribonucleotide monomers.

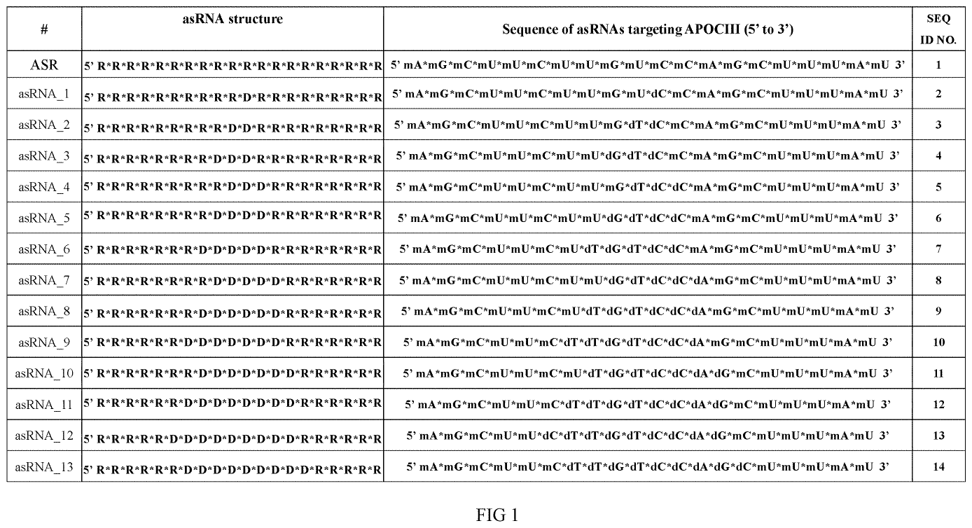

Exemplary structures and sequences of the asRNA molecule of the invention are shown in FIGS. 1, 3 and 5A.

The composition of the invention can be used for modulating gene expression or function in eukaryotic cell in at least three ways: (i) one kind of asRNA molecules are caused to contact a cell or administered to a subject; (ii) different kinds of asRNA molecules are caused to contact a cell or administered to a subject separately at different times; (ii) different kinds of asRNA molecules are caused to contact a cell or administered to a subject simultaneously.

In certain embodiments, the asRNA includes a nucleobase sequence region, called a “targeting region,” that is at least 70%, 75%, 80%, 85%, 86%, 87%, 88%, 89%, 90%, 91%, 92%, 93%, 94%, 95%, 96%, 97%, 98%, 99%, or 100% complementary to the target segment of a target gene to which it is targeted, including an mRNA and a non-coding RNA. In certain embodiments, the asRNA molecule has a nucleobase sequence comprising a fully complementary sequence of the target segment of a target gene to which it is targeted. In certain embodiments, the asRNA molecules has a nucleobase sequence comprising no more than 1, 2 or 3 mismatch(es) when hybridized to the target segment of a target gene to which it is targeted. In certain embodiments, the target gene is selected from mRNA or non-coding RNA that are implicated in a mammalian disease. In some embodiments, at least one ISD is disposed in a targeting region of the asRNA. In certain embodiments, an ISD is positioned at or near the 5′ end of the asRNA, or at or near the 3′ end of the strand. In other embodiments, an ISD is positioned at a more central part (i.e., at least 1, 2, 3, 4, 5, 6, 7, 8, 9, or 10 nucleobases away from both ends, i.e., starting from position no. 2 or more central counting from the end) of the asRNA. In some embodiments, at least one ISD can be positioned at any position of the asRNA.

In various embodiments, the asRNA has a backbone length of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 linked nucleotide monomers, or equivalents thereof, or of a range bracketed by any two of the above values (both range endpoints included). For example, some of the ranges of the length of the asRNA strand include: 8-48 nucleotide monomers; 8-44 nucleotide monomers; 8-42 nucleotide monomers; 8-40 nucleotide monomers; 8-36 nucleotide monomers; 8-33 nucleotide monomers; 10-48 nucleotide monomers; 10-44 nucleotide monomers; 10-42 nucleotide monomers; 10-40 nucleotide monomers; 10-36 nucleotide monomers; 10-30 nucleotide monomers; 10-29 nucleotide monomers; 12-48 nucleotide monomers; 12-44 nucleotide monomers; 12-42 nucleotide monomers; 12-40 nucleotide monomers; 12-36 nucleotide monomers; 12-34 nucleotide monomers; 12-32 nucleotide monomers; 12-30 nucleotide monomers; 12-29 nucleotide monomers; 12-28 nucleotide monomers; 12-26 nucleotide monomers; 12-25 nucleotide monomers; 13-48 nucleotide monomers; 13-44 nucleotide monomers; 13-42 nucleotide monomers; 13-40 nucleotide monomers; 13-36 nucleotide monomers; 13-34 nucleotide monomers; 13-32 nucleotide monomers; 13-30 nucleotide monomers; 13-28 nucleotide monomers; 13-26 nucleotide monomers; 13-25 nucleotide monomers; 13-24 nucleotide monomers; 13-23 nucleotide monomers; 14-36 nucleotide monomers; 15-23 nucleotide monomers; 20-36 nucleotide monomers; 21-36 nucleotide monomers; 24-36 nucleotide monomers; at least 21 nucleotide monomers; at least 24 nucleotide monomers and at least 8 nucleotide monomers.

In certain embodiments, the asRNA is 8 to 36 (both range endpoints included) nucleotide monomers in length. In other words, the asRNAs are from 8 to 36 (both range endpoints included) linked nucleobase monomers. In certain embodiments, the asRNA consists of 20-36 (both range endpoints included) linked nucleoside monomers. In other embodiments, the asRNA comprises an oligonucleotide consisting of 8 to 100, 10 to 80, 12 to 50, 14 to 30, 15 to 23, 16 to 22, 16 to 21, or 20 (both range endpoints included) linked nucleobases.

In the asRNA molecule of the invention, at least one nucleotide monomer can be a modified nucleotide or nucleotide analogue, e.g., a sugar-, backbone-, and/or base-modified nucleotide. In an embodiment, such a backbone-modified nucleotide has at least a modification in an internucleoside linkage, e.g., to include at least one of a nitrogen or sulphur heteroatom. In some embodiments, the modified internucleoside linkage is or includes: phosphorothioate (P═S) group, phosphotriesters, methylphosphonates, or phosphoramidate.

In certain embodiments, the asRNA comprises at least one modified internucleoside linkage. Such modified internucleoside linkage may be between two deoxyribonucleoside monomers, two ribonucleoside monomers, or one deoxyribonucleoside monomer and one ribonucleoside monomer. Alternately, the phosphate group on at least one of the terminal nucleoside monomers may be modified. In certain embodiments, the internucleoside linkage is a phosphorothioate internucleoside linkage. In certain embodiments, the internucleoside linkage is a thio-phosphoramidate internucleoside linkage. In certain embodiments, each internucleoside linkage of the oligonucleotide strand is a phosphorothioate internucleoside linkage. In certain embodiments, all the internucleoside linkages in the asRNA are phosphorothioate internucleoside linkages, or a mixture of phosphorothioate and phosphodiester linkages.

In certain embodiments, the asRNA includes at least one nucleoside monomer having a modified sugar moiety. Such a nucleoside monomer can be a deoxyribonucleoside monomer or a ribonucleoside monomer.

In certain embodiments, the 2′ position of the modified sugar moiety is replaced by a group selected from OR, R, halo, SH, SR, NH₂, NHR, NR₂, or CN, where each R is independently C₁-C₆ alkyl, alkenyl or alkynyl, and halo is F, Cl, Br or I. In some embodiments, the 2′ position of the modified sugar moiety is replaced by a group selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), or O—CH₂—C(═O)—N(R₁)—(CH₂)₂—N(Rₘ)(Rₙ), where each Rₗ, Rₘ and Rₙ is, independently, H or substituted or unsubstituted C₁-C₁₀ alkyl. In some embodiments, the modified sugar moiety is selected from the group of 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. In some embodiments, the modified sugar moiety is substituted by bicyclic sugar selected from the group of 4′-(CH₂)—O-2′ (LNA); 4′-(CH₂)—S-2; 4′-(CH₂)₂—O-2′ (ENA); 4′-CH(CH₃)—O-2′ (cEt) and 4′-CH(CH₂OCH₃)—O-2′, 4′-C(CH₃)(CH₃)—O-2′, 4′-CH₂—N(OCH₃)-2′, 4′-CH₂—O—N(CH₃)-2′, 4′-CH₂—N(R)—O-2′ (where R is H, C₁-C₁₂ alkyl, or a protecting group), 4′-CH₂—C(H) (CH₃)-2′, and 4′-CH₂—C—(—CH₂)-2′.

In some embodiments, the modified sugar moiety is selected from the group of 2′-O-methoxyethyl modified sugar (MOE), a 4′-(CH₂)—O-2′ bicyclic sugar (LNA), 2′-deoxy-2′-fluoroarabinose (FANA), and a methyl(methyleneoxy) (4′-CH(CH₃)—O-2) bicyclic sugar (cEt).

In some embodiments, the asRNA of the invention includes at least one nucleoside monomer having a modified nucleobase. Such a nucleoside monomer can be a deoxyribonucleoside monomer or a ribonucleoside monomer.

In some embodiments, the modified nucleobase is selected from the group of: 5-methylcytosine (5-Me-C), inosine base, a tritylated base, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 1-methyl-pseudo-uracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, and 7-deazaguanine and 7-deazaadenine and 3-deazaguanine and 3-deazaadenine.

In a particular embodiment, the modified nucleobase in the molecule of the invention is a 5-methylcytosine. In an embodiment, each cytosine base in the molecule of the invention is 5-methylcytosine. In certain embodiments, the modified nucleobase is a 5-methyluracil. In certain embodiments, each uracil is a 5-methyluracil.

In a feature, in the molecule of the invention, the asRNA comprise linked ribonucleoside monomers, in addition to the linked ribonucleoside monomers, further includes an ISD that consists of one or more linked deoxyribonucleoside monomers. Further, there may be even more ISD segments. The ISD can be anywhere in the asRNA. In some embodiments, one or more ISDs include a terminal nucleoside monomer, or a penultimate terminal nucleoside monomer. In some embodiments, one or more ISDs are inserted in a segment of ribonucleoside monomers, separating them into multiple segments. In certain embodiments, each of the ISDs independently consists of 1, 2, 3, 4, 5, 6, 7, 8, 9 or 10 linked ribonucleoside monomers.

In certain embodiments, at least one or each of the linked deoxyribonucleoside monomers in the ISDs is a modified deoxyribonucleotide or deoxyribonucleotide analog. The deoxyribonucleotide may be modified in the same or a similar way as follows: have a modified internucleoside linkage, a modified sugar moiety and/or a modified nucleobase.

In some embodiments, the asRNA molecule of the present invention may include at least one CpG motif that can be recognized by the pattern recognition receptors (PRR), e.g., Toll-like receptors.

In some embodiments, the sugar moiety of the deoxyribonucleotide monomer in the asRNA molecule is either the sugar moiety of a naturally occurring deoxyribonucleotide (2-H) or 2′-deoxy-2′-fluoroarabinose (FANA).

In some embodiments, the sugar moiety of the ribonucleotide monomer in the asRNA molecule is selected from the group of a naturally occurring ribonucleotide (2-OH), 2′-F modified sugar, 2′-OMe modified sugar, 2′-O-methoxyethyl modified sugar (MOE), a 4′-(CH₂)—O-2′ bicyclic sugar (LNA) and a methyl(methyleneoxy) (4′-CH(CH₃)—O-2) bicyclic sugar (cEt).

In certain embodiments, each ribonucleoside monomer of the asRNA molecule has a 2′-O-methoxyethyl modified sugar, where each cytosine is a 5-methylcytosine, where each uracil is a 5-methyluracil, or methyl-pseudouracil, and where each internucleoside linkage is a phosphorothioate linkage. In certain embodiments, each deoxyribonucleoside monomer in the ISD has a modified sugar moiety of 2′-deoxy-2′-fluoroarabinose (FANA), where each cytosine is a 5-methylcytosine, and where each internucleoside linkage is a phosphorothioate linkage.

In certain embodiments, the molecule of the invention can be stabilized against degradation, either through at least one chemical modification or a secondary structure. Not only can any or all of the nucleotide monomers in the asRNA chemically modified, it may be conjugated to one or more moieties or ligands to enhance its functionality, for example, with moieties or ligands selected from: peptide, antibody, antibody fragment, polymer, polysaccharide, lipid, hydrophobic moiety or molecule, cationic moiety or molecule, lipophilic compound or moiety oligonucleotide, cholesterol, GalNAc and aptamer.

In certain embodiments, the targeting region of the molecule of the invention does not contain any mismatch or bulge, and is perfectly complementary to the target oligonucleotide in the targeting region. In another embodiment, the targeting region of the asRNA contains mismatch and/or bulge when hybridized with the target RNA. In an embodiment, the entire asRNA is completely complementary to the target RNA.

As is well known to one skilled in the art, it is possible to introduce mismatch bases without eliminating activity. Similarly, the asRNA of the present invention can include unmatched or mismatched region(s) when base pairing with the targeted RNA. Mismatches in asRNA are sometimes desired for reducing off-target effects or enable other features to the asRNA.

In certain embodiments, the target is mRNA or non-coding RNA implicated in a mammalian disease. In certain embodiments, the target is mRNA. In certain embodiments, the target is non-coding RNA, such as microRNA and lncRNA. The asRNA can occupy the target by hybridizing to the target sequence as long as they are substantially complementary to each other, and inactive the target gene.

### 3. MODIFICATIONS

A nucleoside monomer is a base-sugar composition. The nucleobase (also known as base) portion of the nucleoside monomer is normally a heterocyclic base moiety. Nucleotide monomers are nucleoside monomers that further include a phosphate group covalently linked to the sugar portion of the nucleoside. For those nucleoside monomers that include a pentofuranosyl sugar, the phosphate group can be linked to the 2′, 3′ or 5′ hydroxyl moiety of the sugar. Oligonucleotides are formed through the covalent linkage of adjacent nucleoside monomers to one another, to form a linear polymeric oligonucleotide. Within the oligonucleotide structure, the phosphate groups are commonly referred to as forming the internucleoside linkages of the oligonucleotide.

Modifications to the asRNA molecule of the invention encompass substitutions or changes to internucleoside linkages, sugar moieties, or nucleobases. Modified asRNAs are in some cases preferred over native forms because of desirable properties such as, for example, increased inhibitory activity, enhanced cellular uptake, enhanced strand affinity, solubility, reduce the non-specific interaction and resistance to RNase degradation or enhanced stability otherwise. Consequently, comparable results can often be obtained with short asRNA that have such chemically modified nucleoside monomers. One or more of the natural nucleotides in the asRNA of the invention can be substituted with modified nucleotides or nucleotide analogues. The substitution can take place anywhere in the asRNA.

The modifications of oligonucleotide molecules have been investigated to improve the stability of various oligonucleotide molecules, including antisense oligonucleotide, ribozyme, aptamer, and RNAi (Chiu and Rana, 2003; Czauderna et al., 2003; de Fougerolles et al., 2007; Kim and Rossi, 2007; Mack, 2007; Zhang et al., 2006; Schrnidt, 2007; Setten R L et al., 2020; Crooke S T et al., 2018; and Roberts T C et al., 2020).

Any stabilizing modification known to a person skilled in the art can be used to improve the stability of the oligonucleotide molecules. Within the oligonucleotide molecules, chemical modifications can be introduced to the phosphate backbone (e.g., phosphorothioate linkages), the sugar (e.g., locked nucleic acids, glycerol nucleic acid, cEt, 2′-MOE, 2′-fluorouridine, 2′-O-methyl), and/or the base (e.g., 2′-fluoropyrimidines).

Several examples of such chemical modifications are summarized in the sections that follow.

In various embodiment, the modified nucleotide or a nucleotide analogue is sugar-, backbone- and/or base-modified nucleotide.

### 3.1 Modified Internucleoside Linkages or Backbone-Modified Nucleotide

The naturally occurring internucleoside linkage of RNA and DNA is a 3′ to 5′ phosphodiester linkage. The asRNA molecule of the invention having one or more modified, i.e., non-naturally occurring, internucleoside linkages in one or both of its strands are sometimes selected over a corresponding molecule with only naturally occurring internucleoside linkages because of desirable properties such as, for example, enhanced cellular uptake, enhanced affinity for target nucleic acids, and increased stability in the presence of nucleases.

asRNA having modified internucleoside linkages include internucleoside linkages that retain a phosphorus atom as well as internucleoside linkages that do not have a phosphorus atom. In an embodiment, the phosphodiester internucleoside linkage is modified to include at least a nitrogen and/or sulphur heteroatom. Representative phosphorus containing internucleoside linkages include, but are not limited to, phosphodiesters, phosphotriesters, methylphosphonates, phosphoramidate, thio-phosphoramidate and phosphorothioates. Methods of preparation of phosphorous-containing and non-phosphorous-containing linkages are well known.

In one embodiment, a modified nucleotide or nucleotide analogue is a backbone-modified nucleotide. The backbone-modified nucleotide may have a modification in a phosphodiester internucleoside linkage. In a further embodiment, the backbone-modified nucleotide is phosphorothioate internucleoside linkage. In certain embodiments, each internucleoside linkage is a phosphorothioate internucleoside linkage.

### 3.2 Modified Sugar Moieties

The asRNA of the invention can optionally contain one or more nucleoside monomers where the sugar group has been modified. Such sugar-modified nucleoside monomers may impart enhanced nuclease stability, increased binding affinity, or some other beneficial biological property to the strand. In certain embodiments, nucleoside monomers comprise chemically modified ribofuranose ring moieties. Examples of chemically modified ribofuranose rings include without limitation, addition of substitute groups (including 5′ and 2′ substituent groups, bridging of non-geminal ring atoms to form bicyclic nucleic acids (BNA), replacement of the ribosyl ring oxygen atom with S, N(R), or C(R₁)(R₂)(R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′, 2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleoside monomers having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each Rl, Rm and Rn is, independently, H or substituted or unsubstituted C₁-C₁₀ alkyl.

Bicyclic nucleosides are modified nucleosides having a bicyclic sugar moiety. Examples of bicyclic nucleic acids (BNAs) include without limitation nucleosides comprising a bridge between the 4′ and the 2′ ribosyl ring atoms. In certain embodiments, the asRNA provided herein include one or more BNA nucleosides wherein the bridge comprises one of the formulas: 4′-(CH₂)—O-2′ (LNA); 4′-(CH₂)—S-2; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see PCT/US2008/068922 published as WO/2009/006478, published Jan. 8, 2009); 4′-CH₂—N(OCH₃)-2′ (and analogs thereof see PCT/US2008/064591 published as WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H) (CH₃)-2′ (see Chattopadhyaya et al., J. Org. Chem., 2009, 74, 118-134); and 4′-CH₂—C—(═CH₂)-2′ (and analogs thereof see PCT/US2008/066154 published as WO 2008/154401, published on Dec. 8, 2008).

In certain embodiments, bicyclic nucleosides include, but are not limited to, (A) α-L-methyleneoxy (4′-CH₂—O-2) BNA (B) β-D-methyleneoxy (4′-CH₂—O-2) BNA (C) ethyleneoxy (4′-(CH₂)₂—O-2′) BNA, (D) aminooxy (4′-CH₂—O—N(R)-2′) BNA, (E) oxyamino (4′-CH₂—N(R)—O-2) BNA, (F) methyl(methyleneoxy) (4′-CH(CH₃)—O-2) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2) BNA, (J) propylene carbocyclic (4′-(CH₂)₃-2′) BNA, and (K) vinyl BNA.

In certain embodiments, a modified nucleotide or a nucleotide analogue is a sugar-modified ribonucleotide, in which the 2′-OH group is replaced by a group selected from: H, OR, R, halo, SH, SR, NH₂, NHR, NR₂, and CN, where each R is independently selected from the group consisting of: C₁-C₆ alkyl, alkenyl and alkynyl, and halo is selected from the group of F, Cl, Br and I. In certain embodiments, the sugar-modified ribonucleotide is selected from the group of 2′-OMe modified nucleotide, 2′-F modified nucleotide, 2′-O-methoxyethyl(2′MOE) modified nucleotide, LNA (Locked nucleic acid) modified nucleotide, GNA (Glycerol nucleic acid) modified nucleotide, and cEt (Constrained ethyl) modified nucleotide.

Chemical modifications at the 2′ position of the ribose, such as 2′-O-methylpurines and 2′-fluoropyrimidines, which increases resistance to endonuclease activity in serum, can be adopted to stabilize the molecules of the present invention. The position for the introduction of the modification should be carefully selected to avoid significantly reducing the silencing/regulating of potency of the molecule. In certain embodiments, the first nucleotide monomer adjacent to the 5′-terminal nucleotide monomer of the strand is a 2′-flouro-ribonucleotide.

### 3.3 Modified Nucleobases

The asRNA can also have nucleobase (or base) modifications or substitutions. Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to the asRNA molecule. Modified nucleobases include synthetic and natural nucleobases such as, for example, 5-methylcytosine (5-Me-C). Certain nucleobase substitutions, including 5-methylcytosine substitutions, are particularly useful for increasing the binding affinity of the antisense strand. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include and are not limited to: 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 1-methyl pseudouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine.

Heterocyclic base moieties may include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of an antisense strand include 5-substituted pyrimidines, 6-azapyrimidines and N-2, N-6 and O-6 substituted purines, including 2 aminopropyladenine, 5-propynyluracil and 5-propynylcytosine.

In certain embodiments, a modified nucleotide or a nucleotide analogue is a base-modified nucleotide. In an embodiment, a modified nucleotide or a nucleotide analogue has an unusual base or a modified base. In certain embodiments, the modified base is a 5-methylcytosine (5′-Me-C). In certain embodiments, each cytosine is a 5-methylcytosine. In certain embodiments, the modified base is a 5-methyluracil (5′-Me-U). In certain embodiments, each uracil is a 5-methyluracil.

Any modified nucleotide or analogue that may benefit the stability or affinity can be made without departing from the spirit and scope of the present invention. Several examples of such chemical modifications are same as summarized above.

### 4. PHARMACEUTICAL COMPOSITION

In some embodiments, the present invention also provides pharmaceutical formulations comprising the asRNA of the present invention, or a pharmaceutically acceptable derivative thereof and at least one pharmaceutically acceptable excipient or carrier. As used herein, “pharmaceutically acceptable excipient” or “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in “Remington: The Science and Practice of Pharmacy, Twentieth Edition,” Lippincott Williams & Wilkins, Philadelphia, PA., which is incorporated herein by reference. Examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the asRNA molecule, use thereof in the compositions is contemplated.

Examples of the pharmaceutically acceptable carrier that can be used with the molecule of the invention include but are not limited to: a pharmaceutical carrier, a positive-charge carrier, a liposome, a lipid nanoparticle, a protein carrier, a hydrophobic moiety or molecule, a cationic moiety or molecule, GalNAc, a polysaccharide a polymer, a nanoparticle, a nanoemulsion, a cholesterol, a lipid, a lipophilic compound or moiety, and a lipoid.

In a certain embodiment, the present invention provides a method of treatment comprising administering a therapeutically effective amount of the pharmaceutical composition to a subject in need thereof. In an embodiment, the pharmaceutical composition is administered via a route selected from the group of: intravenous injection (iv), subcutaneous injection (sc), per os (po), intramuscular (im) injection, oral administration, inhalation, topical, intrathecal, and other regional administrations. In another embodiment, the therapeutically effective amount is 1 ng to 1 g per day, 100 ng to 1 g per day, or 1 μg to 1000 mg per day.

Methods for formulation are disclosed in PCT International Application PCT/US02/24262 (WO03/01 1224), U.S. Patent Application Publication No. 2003/0091639 and U.S. Patent Application Publication No. 2004/0071775, each of which is incorporated by reference herein.

An asRNA molecule of the present invention is administered in a suitable dosage form prepared by combining a therapeutically effective amount (e.g., an efficacious level sufficient to achieve the desired therapeutic effect through inhibition of tumor growth, killing of tumor cells, treatment or prevention of cell proliferative disorders, etc.) of the asRNA molecule of the present invention (as an active ingredient) with standard pharmaceutical carriers or diluents according to conventional procedures (i.e., by producing a pharmaceutical composition of the invention).

These procedures may involve mixing, granulating, and compressing or dissolving the ingredients as appropriate to attain the desired preparation. In another embodiment, a therapeutically effective amount of asRNA molecules is administered in a suitable dosage form without standard pharmaceutical carriers or diluents. In some embodiments, a therapeutically effective amount of the molecule of the invention is administered in a suitable dosage form. Pharmaceutically acceptable carriers include solid carriers such as lactose, terra alba, sucrose, talc, gelatin, agar, pectin, acacia, magnesium stearate, stearic acid and the like. Exemplary liquid carriers include syrup, peanut oil, olive oil, water and the like. Similarly, the carrier or diluent may include time-delay material known in the art, such as glyceryl monostearate or glyceryl distearate, alone or with a wax, eihylcellulose, hydroxypropylmethylcellulose, methylmethacrylate or the like. Other fillers, excipients, flavorants, and other additives such as are known in the art may also be included in a pharmaceutical composition according to this invention.

The pharmaceutical compositions of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers comprising excipients and/or auxiliaries which facilitate processing of the antisense oligonucleotide into preparations that can be used pharmaceutically. Of course, the appropriate formulation is dependent upon the route of administration chosen.

The composition, compound, combination or the pharmaceutical composition of the invention can be administered to a subject in many of the well-known methods currently used for chemotherapeutic treatment. For example, for treatment of cancers, the asRNA molecule of the invention may be injected directly into tumors, injected into the blood stream or body cavities or taken orally or applied through the skin with patches. For treatment of psoriatic conditions, systemic administration (e.g., oral administration), or topical administration to affected areas of the skin, are preferred routes of administration. The dose chosen should be sufficient to constitute effective treatment but not as high as to cause unacceptable side effects. The state of the disease condition (e.g., cancer, psoriasis, and the like) and the health of the patient should be closely monitored during and for a reasonable period after treatment.

### 5. Utility

### 5.1 Method of Use

The present invention also provides a method of modulating gene expression or function in a cell or an organism. The cell may be a eukaryotic cell, e.g., a mammalian cell. The method comprises the steps of contacting said cell or organism with the asRNA molecule disclosed herein, under conditions wherein selective gene silencing can occur, and mediating a selective gene silencing effected by the asRNA molecule towards a target nucleic acid having a sequence portion substantially complementary to the antisense strand. The target nucleic acid may be an RNA such as a mRNA or non-coding RNA where such RNA either encodes a protein or regulates a part of a biological pathway implicated in a disease.

In an embodiment, the contacting step comprises the step of introducing asRNA molecule into a target cell in culture or in an organism in which the selective gene silencing can occur. In a further embodiment, the introducing step comprises a mixing, transfection, lipofection, infection, electroporation, or other delivery technologies. In another embodiment, the introducing step comprises using a pharmaceutically acceptable excipient, carrier, or diluent selected from the group of a pharmaceutical carrier, a positive-charge carrier, a liposome, a lipid nanoparticle, a protein carrier, a polymer, a nanoparticle, a nanoemulsion, a lipid, N-Acetyl-Galactosamine (GalNAc), a lipophilic compound or moiety and a lipoid to be administered via iv, sc, intrathecal, po, inhalation, topical or other clinically acceptable administration methods.

In an embodiment, the silencing method is used for determining the function or utility of a gene in a cell or an organism.

In an embodiment, the gene or RNA targeted by the composition of the invention is associated with or implicated in a disease, e.g., a human disease or an animal disease, a pathological condition, or an undesirable condition. In a further embodiment, the target gene or RNA is that of a pathogenic microorganism. In an even further embodiment, the target gene or RNA is of a viral origin. In another embodiment, the target gene or RNA is tumor-associated.

In an alternative embodiment, the gene or RNA targeted by the composition of the invention is a gene or a RNA associated with, or more specifically, implicated with cancer, autoimmune disease, inflammatory diseases, degenerative diseases, infectious diseases, proliferative diseases, metabolic diseases, immune-mediated disorders, allergic diseases, dermatological diseases, malignant diseases, gastrointestinal disorders, hepatic disorders, respiratory disorders, cardiovascular disorders, dermatological disorders, renal disorders, rheumatoid disorders, neurological disorders, psychiatric disorders, endocrine disorders, or aging-related disorders or diseases.

### 5.2 Treatment Method

The present invention also provides a method of treating or preventing various diseases or conditions, including those summarized for ASO and siRNAs (Czech, 2006; de Fougerolles et al., 2007; Dykxhoorn et al., 2003; Kim and Rossi, 2007; Mack, 2007; Crooke S T et al., 2018; Setten R L et al., 2019; Roberts T C et al., 2020). The method comprises administering an effective amount of the asRNA molecule to a subject in need thereof under conditions wherein desired gene inhibition described in the section immediately above can occur.

In an exemplary embodiment, a pharmaceutical composition having the asRNA molecule and a pharmaceutically acceptable excipient, carrier, or diluent is administered to a patient in need thereof for treating or preventing a disease or an undesirable condition in a therapeutically effective amount.

In some embodiments, the present invention can be used as a cancer therapy or to prevent cancer. The composition of the asRNA can be used to silence or knock down genes involved with cell proliferation or other cancer phenotypes. Examples of these genes are k-Ras, β-catenin, Stat3. These oncogenes are implicated in a large number of cancer types.

The novel composition of the invention can also be used to treat or prevent ocular disease, (e.g., age-related macular degeneration (AMD) and diabetic retinopathy (DR)); infectious diseases (e.g., HIV/AIDS, hepatitis B virus (HBV), hepatitis C virus (HCV), human papillomavirus (HPV), herpes simplex virus (HSV), RCV, cytomegalvirus (CMV), dengue fever, west Nile virus); respiratory disease (e.g., respiratory syncytial virus (RSC), asthma, cystic fibrosis); neurological diseases (e.g., Huntingdon's disease (HD), amyotrophic lateral sclerosis (ALS), spinal cord injury, Parkinson's disease, Alzheimer's disease, pain); cardiovascular diseases; metabolic disorders (e.g., hyperlipidemia, hypercholesterolemia, and diabetes); genetic disorders; and inflammatory conditions (e.g., inflammatory bowel disease (IBD), arthritis, rheumatoid disease, autoimmune disorders), dermatological diseases.

In an alternative embodiment, the administration method is a route selected from the group of intravenous injection (iv), subcutaneous injection (sc), per os (po), intrathecal, inhalation, topical, and regional administration.

### EXAMPLES

Examples are provided below to further illustrate different features of the present invention. The examples also illustrate useful methodology for practicing the invention. These examples do not limit the claimed invention.

### Methods and Materials

### Cell Culture

HepaRG cells were grown in William's Medium supplemented with 10% FBS, 10 mg/ml Hydrocortisone, and 4 mg/ml human recombinant insulin. Other proper commercially available cell lines can be purchased and used as known to a person skilled in the art.

Transfection of asRNAs to Cells

24 hours before transfection, the HepaRG cells or other commonly used cell lines were seeded to 6-well plates (1×105 cells/2 mL/well). The asRNAs were transfected by Lipofectamine® RNAiMAX (Thermo Fisher, USA) at different final concertation, such as 100 μM or 10 nM final concentrations as described the manufacture methods, briefly asRNAs and RNAiMAX were incubate for 20 minutes in serum free OPTI-MEM (Thermo Fisher), then added to the cell with culture medium.

### Quantitative PCR

Cells transfected with the indicated asRNAs were harvested at 48 hours after transfection. RNA was isolated with TRIZOL, and qRT-PCR performed using TaqMan one-step RT-PCR reagents and APOCIII assay for APOCIII mRNA detection, etc. The gene GAPDH mRNA levels were used as internal control.

### Target Sequence

To investigate the gene silencing effects of the asRNA disclosed in the present invention, asRNA were designed and made to target different genes. The target gene, target sequence and sequence of exemplary asRNA designed and used are listed below in Table 1.

TABLE 1
Target sequence and sequence of exemplary asRNAs used in below examples

| Target | Accession | Target | SEQ | Antisense | SEQ |
|---|---|---|---|---|---|
| gene | # | sequence 5′ to 3′ | ID NO. | sequence 5′ to 3′ | ID NO. |
| APOCIII | NM_000040 | ATAAAGCTGGACAAGAAGCT | 36 | AGCUUCUUGUCCAGCUUUAU | 37 |

### Example 1: Structure-Activity Relationship (SAR) on asRNA with Various ISDs

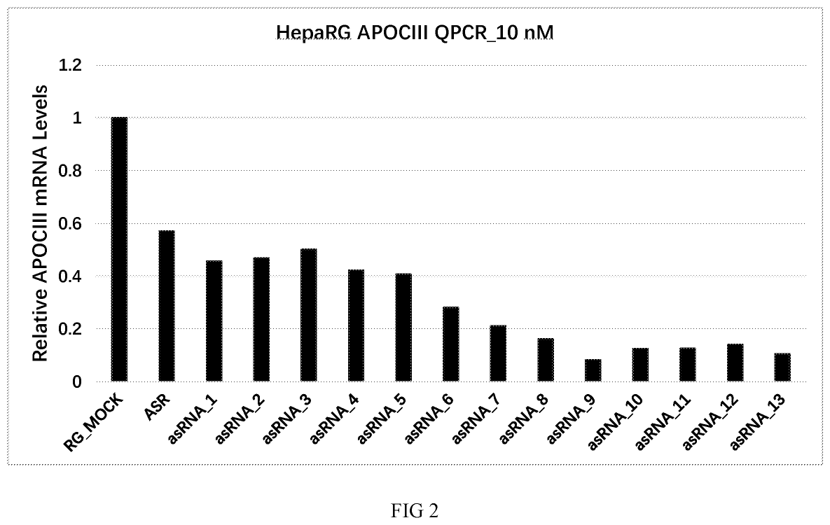

FIG. 1 shows various structural designs of a series of embodiments of asRNAs with various ISDs (labelled as asRNA_1-13). asRNA_1-13 for targeting the APOCIII gene were designed. Corresponding antisense single-stranded RNAs without ISD (ASR) are also designed to be used for comparison (structure and sequence are shown in FIG. 1.) The gene-silencing activities of these asRNAs and the corresponding ASR were tested in HepaRG cells at 10 nM (FIG. 2).

In FIG. 1, all Letters “D” in the illustrated structures represent DNA residues or deoxyribonucleotide monomers; all Letters “R” in the illustrated structures represent RNA residues or ribonucleotide monomers. All Letters “dA, dT, dG, dC” in the sequences represent DNA residues; all Letters “mA, mU, mG, mC” in the sequences represent 2′-MOE modified RNA residues; all “*” in the illustrated structures and the sequences represent PS (phosphorothioate internucleoside linkage).

The results suggest that the gene silencing activity of all designed asRNAs with at least one ISD are more potent as well as more efficacious than the corresponding ASR without ISD.

### Example 2: SAR on asRNA with Various Positions of ISD

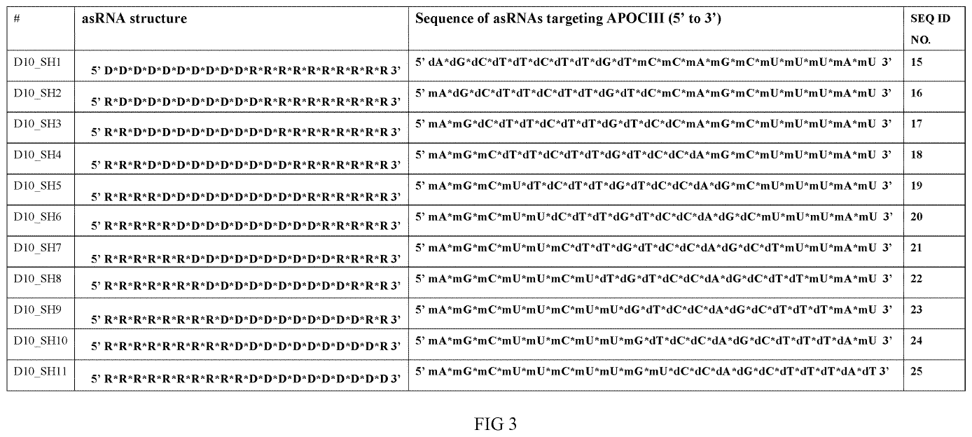

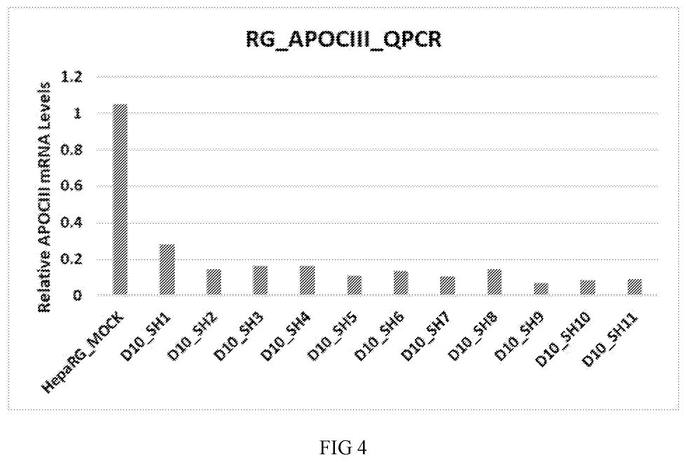

FIG. 3 shows different structural designs of another series of embodiments of asRNAs by holding constant the length of the asRNA and the number of deoxyribonucleotide monomers of ISD while changing the position of the ISD (labeled as D10-SH1~D10-SH11). Exemplary sequences of the asRNA for targeting the APOCIII gene are also shown in FIG. 3. The gene silencing activities of these asRNAs D10-SH1~D10-SH11 for targeting the APOCIII gene were tested in HepaRG Cells at 10 nM and results are showed in FIG. 4.

In FIG. 3, all Letters “D”, “R”, “dA, dT, dG, dC”, “mA, mU, mG, mC” and all “*” in the illustrated structures and sequences represent the same as in FIG. 1.

The results suggest that all designed asRNAs with various positions of ISD have potent gene silencing activity.

### Example 3: SAR on asRNA with Various Length

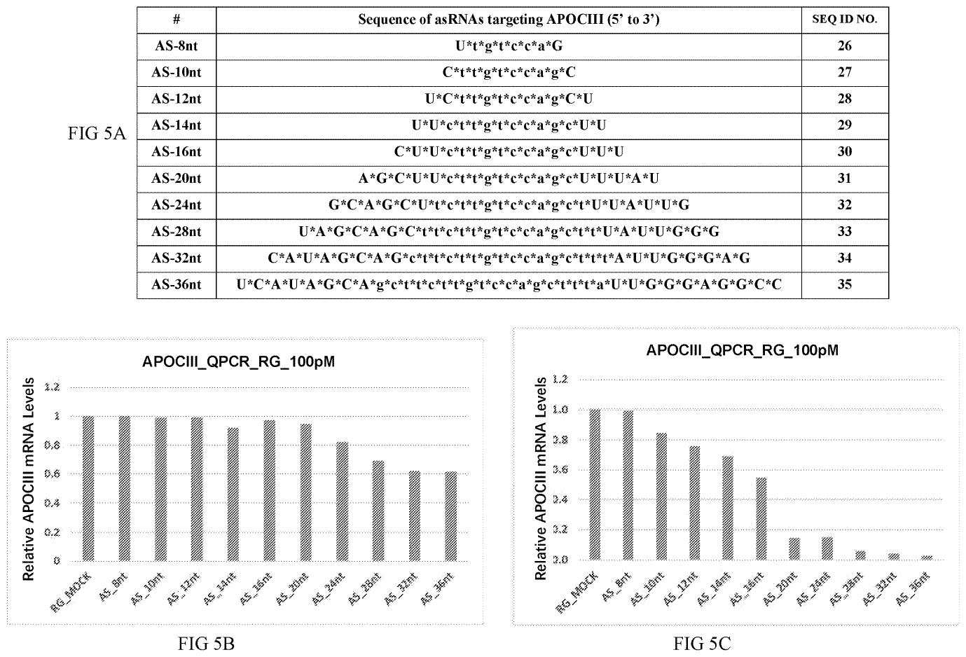

FIG. 5A shows different structural designs of another series of embodiments of asRNAs. In these asRNAs, various length of asRNA molecule for targeting the APOCIII gene were designed (labeled as AS-8nt to AS-36nt, structures and sequences are shown in FIG. 5A). Gene silencing activities of the asRNAs of 8-36 nt in length were designed to target APOCIII gene. The gene silencing activities of these asRNAs were tested in HepaRG Cells at 100 μM and 10 nM concentrations (FIG. 5B and FIG. 5C).

In FIG. 5A, all lowercase Letters “a, c, g, t” represent DNA residues; all uppercase Letters “A, C, G, U” represent 2′-MOE modified RNA residues, wherein all “U” is 5-Methyl Uridine 2′-MOE RNA residues; wherein all “C” and “c” are 5-Me-C; all “*” represent PS (phosphorothioate internucleoside linkage).

The results suggest that all designed asRNAs with at least 10 linked nucleotide monomers in length have potent gene silencing activity. It is a surprising discovery that longer asRNAs are more potent in gene silencing activity with asRNAs of 20 nt or longer being especially more potent. In contrast, the typical length for ASO is 16-20 nt. asRNAs of longer than 20 nt are much more potent than typical ASO that was optimized with the most advanced state-of-art knowhows.

The results in Examples 1-3 strongly suggest that the asRNA with ISD designed according to the principles of the present invention can achieve great potency of gene silencing.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### REFERENCE

1. Xu J Z, Zhang J L, Zhang W G. Antisense RNA: the new favorite in genetic research. J Zhejiang Univ Sci B. 2018 October; 19(10):739-749. doi: 10.1631/jzus.B1700594. PMID: 30269442; PMCID: PMC6194357.
2. C. Frank Bennett and Eric E. Swayze, RNA Targeting Therapeutics: Molecular Mechanisms of Antisense Oligonucleotides as a Therapeutic Platform. Annu. Rev. Pharmacol. Toxicol. 2010. 50:259-93.
3. C. Frank Bennett. Therapeutic Antisense Oligonucleotides Are Coming of Age. Annu Rev Med. 2019 Jan. 27; 70:307-321. doi: 10.1146/annurev-med-041217-010829. PMID: 30691367.
4. Crooke S T, Witztum J L, Bennett C F, Baker B F. RNA-Targeted Therapeutics. Cell Metab. 2018 Apr. 3; 27(4):714-739. doi: 10.1016/j.cmet.2018.03.004. Erratum in: Cell Metab. 2019 Feb. 5; 29(2):501. PMID: 29617640.
5. Roberts T C, Langer R, Wood M J A. Advances in oligonucleotide drug delivery. Nat Rev Drug Discov. 2020 October; 19 (10):673-694. doi: 10.1038/s41573-020-0075-7. Epub 2020 Aug. 11. PMID: 32782413; PMCID: PMC7419031.
6. de Fougerolles A, Vornlocher H P, Maraganore J, Lieberman J. Interfering with disease: a progress report on siRNA-based therapeutics. Nature Rev Drug Discov. 2007; 6:443-453. [PubMed: 17541417]

1. A short antisense RNA (asRNA) molecule comprising a single strand of linked nucleotide monomers,
wherein the strand is substantially complementary to a targeted segment of a targeted RNA through at least one targeting region, and has a length selected from the group consisting of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 nucleotide monomers; and
wherein the asRNA molecule comprises at least one interspersed segment of deoxyribonucleotide monomer(s) (ISD) that each ISD, independently of each other, comprises either one deoxyribonucleotide monomer, or at least 2, 3, 4, 5, 6, 7 or 8 contiguous one deoxyribonucleotide monomer.

2. The asRNA molecule of claim 1, wherein the asRNA has at least one improved gene modulation property or pharmaceutical property compared to a corresponding single-stranded antisense RNA without ISD.

3. (canceled)

4. (canceled)

5. (canceled)

6. The asRNA molecule of claim 1, wherein the strand is at least 70%, 80%, 85%, 90%, 95% complementary or fully complementary to the targeted segment of the targeted RNA.

7. (canceled)

8. The asRNA molecule of claim 1, wherein the asRNA has a length selected from the group consisting of:
(a) 8-50 nucleotide monomers,
(b) 10-48 nucleotide monomers,
(c) 10-36 nucleotide monomers,
(d) 12-36 nucleotide monomers,
(e) 12-25 nucleotide monomers, and
(f) 21-36 nucleotide monomers.

9. The asRNA molecule of claim 1, wherein at least one nucleotide monomer of the asRNA is a modified nucleotide or nucleotide analogue; wherein the modified nucleotide or nucleotide analogue is selected from the group consisting of:
(A) a backbone-modified nucleotide;
(B) a sugar moiety-modified nucleotide;
(C) a nucleobase-modified nucleotide; and
(D) a combination thereof.

10. (canceled)

11. The asRNA molecule of claim 9, wherein, in (A), the backbone-modified nucleotide has a modification in an internucleoside linkage, and wherein:
(a) the internucleoside linkage is modified to include at least one of a nitrogen or sulphur heteroatom;
(b) the modified internucleoside linkage has been modified to contain a linkage selected from the group consisting of phosphorothioate (P═S) group, phosphotriesters, methylphosphonates, and phosphoramidate; and/or
(c) the asRNA comprises at least one modified internucleoside linkage, and wherein the modified internucleoside linkage is a phosphorothioate internucleoside linkage.

12. The asRNA molecule of claim 11, wherein, in (c), each internucleoside linkage of the asRNA is a phosphorothioate internucleoside linkage.

13. The asRNA molecule of claim 9, wherein, in (B):
(a) the 2′ position of the modified sugar moiety is replaced by a chemical group selected from the group consisting of OR, R, halo, SH, SR, NH₂, NHR, NR₂, and CN, where each R is independently C₁-C₆ alkyl, alkenyl or alkynyl, and halo is F, Cl, Br or I;
(b) the 2′ position of the modified sugar moiety is replaced by a chemical group selected from the group consisting of allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each of Rₗ, Rₘ and Rₙ is, independently, H or substituted or unsubstituted C₁-C₁₀ alkyl;
(c) the modified sugar moiety is selected from the group consisting of 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups;
(d) the modified sugar moiety is substituted by bicyclic sugar selected from the group consisting of 4′-(CH₂)—O-2′ (LNA); 4′-(CH₂)—S-2; 4′-(CH₂)₂—O-2′ (ENA); 4′-CH(CH₃)—O-2′ (cEt) and 4′-CH(CH₂OCH₃)—O-2′, 4′-C(CH₃)(CH₃)—O-2′, 4′-CH₂—N(OCH₃)-2′, 4′-CH₂—O—N(CH₃)-2′, 4′-CH₂—N(R)—O-2′ (where R is H, C₁-C₁₂ alkyl, or a protecting group), 4′-CH₂—C(H)(CH₃)-2′, and 4′-CH₂—C—(═CH₂)-2′; and/or
(e) the modified sugar moiety is selected from the group consisting of 2′-O-methyl modified sugar, 2′-O-methoxyethyl modified sugar (MOE), a 4′-(CH₂)—O-2′ bicyclic sugar (LNA), 2′-deoxy-2′-fluoroarabinose (FANA), and a methyl(methyleneoxy) (4′-CH(CH₃)—O-2) bicyclic sugar (cEt).

14. The asRNA molecule of claim 9, wherein, in (C):
(a) the modified nucleobase is selected from the group consisting of 5-methylcytosine (5-Me-C), inosine base, a tritylated base, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 1-methyl-pseudo-uracil, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, and 7-deazaguanine and 7-deazaadenine and 3-deazaguanine and 3-deazaadenine;
(b) the modified nucleobase is a 5-methylcytosine; and/or
(c) each cytosine base is 5-methylcytosine.

15. The asRNA molecule of claim 1, wherein the asRNA is used for modulating gene expression or function in a cell; wherein the cell is eukaryotic and mammalian.

16. The asRNA molecule of claim 1, wherein the targeted RNA is selected from the group consisting of:
(a) an mRNA, pre-mRNA, mt-mRNA, or non-coding RNA where such RNA either encodes a protein or regulates a part of a biological pathway implicated in a disease;
(b) an mRNA, pre-mRNA or mt-RNA of a gene implicated in a human or animal disease or condition;
(c) an mRNA or pre-mRNA of a gene of a pathogenic microorganism;
(d) a viral RNA;
(e) a long non-coding RNA (lncRNA);
(f) an miRNA, and
(g) an RNA implicated in a disease or disorder selected from the group consisting of an autoimmune disease, an inflammatory disease, a degenerative disease, an infectious disease, a proliferative disease, a metabolic disease, an immune-mediated disorder, an allergic disease, a dermatological disease, a malignant disease, a gastrointestinal disorder, a respiratory disorder, a cardiovascular disorder, a renal disorder, a rheumatoid disorder, a neurological disorder, an endocrine disorder, and an aging-related disorder.

17. (canceled)

18. The asRNA molecule of claim 1, wherein the asRNA is conjugated to a ligand or a moiety; wherein the ligand or moiety is selected from the group consisting of peptide, antibody, polymer, polysaccharide, lipid, hydrophobic moiety or molecule, cationic moiety or molecule, lipophilic compound or moiety oligonucleotide, cholesterol, GalNAc, and aptamer.

19. (canceled)

20. A pharmaceutical composition comprising an asRNA molecule of claim 1 as an active agent, and a pharmaceutically acceptable excipient, carrier, or diluent.

21. (canceled)

22. A method for treating or preventing a disease or a condition, wherein the method comprises administering a therapeutically effective amount of the asRNA molecule of claim 1 or the pharmaceutical composition of claim 20 to a subject in need thereof.

23. The method of claim 22, wherein the disease or condition is selected from the group consisting of cancer, autoimmune disease, inflammatory diseases, degenerative diseases, infectious diseases, proliferative diseases, metabolic diseases, immune-mediated disorders, allergic diseases, dermatological diseases, malignant diseases, gastrointestinal disorders, hepatic disorders, respiratory disorders, cardiovascular disorders, dermatological disorders, renal disorders, rheumatoid disorders, neurological disorders, psychiatric disorders, endocrine disorders, and aging-related disorders.

24. (canceled)

25. A method for modulating a gene expression or gene function in a eukaryotic cell, wherein the method comprises contacting the cell with an effective amount of the asRNA molecule of claim 1 or the pharmaceutical composition of claim 20.


SEQUENCE LISTING (37 sequences)

SEQ ID NO: 1 - RNA, 20 nt, synthetic construct
  source 1..20: mol_type = other RNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = 2'-MOE guanosine-3'-phosphorothioate
  modified_base 10: mod_base = OTHER; note = 2'-MOE uridine-3'-phosphorothioate
  modified_base 11..12: mod_base = OTHER; note = 2'-MOE Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 2 - DNA, 20 bp, synthetic construct
  misc_feature 1..10: note = RNA
  misc_feature 11: note = DNA
  misc_feature 12..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = 2'-MOE guanosine-3'-phosphorothioate
  modified_base 10: mod_base = OTHER; note = 2'-MOE uridine-3'-phosphorothioate
  modified_base 11: mod_base = OTHER; note = Cytidine-3'-phosphorothioate
  modified_base 12: mod_base = OTHER; note = 2'-MOE Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 3 - DNA, 20 bp, synthetic construct
  misc_feature 1..9: note = RNA
  misc_feature 10..11: note = DNA
  misc_feature 12..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = 2'-MOE guanosine-3'-phosphorothioate
  modified_base 10: mod_base = OTHER; note = thymidine-3'-phosphorothioate
  modified_base 11: mod_base = OTHER; note = Cytidine-3'-phosphorothioate
  modified_base 12: mod_base = OTHER; note = 2'-MOE Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 4 - DNA, 20 bp, synthetic construct
  misc_feature 1..8: note = RNA
  misc_feature 9..11: note = DNA
  misc_feature 12..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11: mod_base = OTHER; note = Cytidine-3'-phosphorothioate
  modified_base 12: mod_base = OTHER; note = 2'-MOE Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 5 - DNA, 20 bp, synthetic construct
  misc_feature 1..9: note = RNA
  misc_feature 10..12: note = DNA
  misc_feature 13..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = 2'-MOE guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 6 - DNA, 20 bp, synthetic construct
  misc_feature 1..8: note = RNA
  misc_feature 9..12: note = DNA
  misc_feature 13..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 7 - DNA, 20 bp, synthetic construct
  misc_feature 1..7: note = RNA
  misc_feature 8..12: note = DNA
  misc_feature 13..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7: mod_base = OTHER; note = 2'-MOE uridine-3'-phosphorothioate
  modified_base 8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 8 - DNA, 20 bp, synthetic construct
  misc_feature 1..8: note = RNA
  misc_feature 9..13: note = DNA
  misc_feature 14..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 9 - DNA, 20 bp, synthetic construct
  misc_feature 1..7: note = RNA
  misc_feature 8..13: note = DNA
  misc_feature 14..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7: mod_base = OTHER; note = 2'-MOE uridine-3'-phosphorothioate
  modified_base 8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 10 - DNA, 20 bp, synthetic construct
  misc_feature 1..6: note = RNA
  misc_feature 7..13: note = DNA
  misc_feature 14..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7: mod_base = OTHER; note = thymidine-3'-phosphorothioate
  modified_base 8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 11 - DNA, 20 bp, synthetic construct
  misc_feature 1..7: note = RNA
  misc_feature 8..14: note = DNA
  misc_feature 15..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7: mod_base = OTHER; note = 2'-MOE uridine-3'-phosphorothioate
  modified_base 8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 12 - DNA, 20 bp, synthetic construct
  misc_feature 1..6: note = RNA
  misc_feature 7..14: note = DNA
  misc_feature 15..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 13 - DNA, 20 bp, synthetic construct
  misc_feature 1..5: note = RNA
  misc_feature 6..14: note = DNA
  misc_feature 15..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 14 - DNA, 20 bp, synthetic construct
  misc_feature 1..6: note = RNA
  misc_feature 7..15: note = DNA
  misc_feature 16..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 15 - DNA, 20 bp, synthetic construct
  misc_feature 1..10: note = DNA
  misc_feature 11..20: note = RNA
  source 1..20: mol_type = other DNA
  modified_base 1: mod_base = OTHER; note = adenosine-3'-phosphorothioate
  modified_base 2: mod_base = OTHER; note = guanosine-3'-phosphorothioate
  modified_base 3: mod_base = OTHER; note = Cytidine-3'-phosphorothioate
  modified_base 4..5: mod_base = OTHER; note = thymidine-3'-phosphorothioate
  modified_base 6: mod_base = OTHER; note = Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = 2'-MOE Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 16 - DNA, 20 bp, synthetic construct
  misc_feature 1: note = RNA
  misc_feature 2..11: note = DNA
  misc_feature 12..20: note = RNA
  source 1..20: mol_type = other DNA
  modified_base 1: mod_base = OTHER; note = 2'-MOE adenosine-3'-phosphorothioate
  modified_base 2: mod_base = OTHER; note = guanosine-3'-phosphorothioate
  modified_base 3: mod_base = OTHER; note = Cytidine-3'-phosphorothioate
  modified_base 4..5: mod_base = OTHER; note = thymidine-3'-phosphorothioate
  modified_base 6: mod_base = OTHER; note = Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11: mod_base = OTHER; note = Cytidine-3'-phosphorothioate
  modified_base 12: mod_base = OTHER; note = 2'-MOE Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 17 - DNA, 20 bp, synthetic construct
  misc_feature 1..2: note = RNA
  misc_feature 3..12: note = DNA
  misc_feature 13..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = Cytidine-3'-phosphorothioate
  modified_base 4..5: mod_base = OTHER; note = thymidine-3'-phosphorothioate
  modified_base 6: mod_base = OTHER; note = Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = 2'-MOE adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 18 - DNA, 20 bp, synthetic construct
  misc_feature 1..3: note = RNA
  misc_feature 4..13: note = DNA
  misc_feature 14..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = thymidine-3'-phosphorothioate
  modified_base 6: mod_base = OTHER; note = Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = 2'-MOE guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 19 - DNA, 20 bp, synthetic construct
  misc_feature 1..4: note = RNA
  misc_feature 5..14: note = DNA
  misc_feature 15..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4: mod_base = OTHER; note = 2'-MOE uridine-3'-phosphorothioate
  modified_base 5: mod_base = OTHER; note = thymidine-3'-phosphorothioate
  modified_base 6: mod_base = OTHER; note = Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = 2'-MOE Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 20 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = Cytidine-3'-phosphorothioate
  modified_base 16..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 21 - DNA, 20 bp, synthetic construct
  misc_feature 1..6: note = RNA
  misc_feature 7..16: note = DNA
  misc_feature 17..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = Cytidine-3'-phosphorothioate
  modified_base 16: mod_base = OTHER; note = thymidine-3'-phosphorothioate
  modified_base 17..18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 22 - DNA, 20 bp, synthetic construct
  misc_feature 1..7: note = RNA
  misc_feature 8..17: note = DNA
  misc_feature 18..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7: mod_base = OTHER; note = 2'-MOE uridine-3'-phosphorothioate
  modified_base 8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = Cytidine-3'-phosphorothioate
  modified_base 16..17: mod_base = OTHER; note = thymidine-3'-phosphorothioate
  modified_base 18: mod_base = OTHER; note = 2'-MOE uridine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 23 - DNA, 20 bp, synthetic construct
  misc_feature 1..8: note = RNA
  misc_feature 9..18: note = DNA
  misc_feature 19..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = Cytidine-3'-phosphorothioate
  modified_base 16..18: mod_base = OTHER; note = thymidine-3'-phosphorothioate
  modified_base 19: mod_base = OTHER; note = 2'-MOE adenosine-3'-phosphorothioate
  modified_base 20: mod_base = OTHER; note = 2'-MOE uridine
agcttcttgtccagctttat

SEQ ID NO: 24 - DNA, 20 bp, synthetic construct
  misc_feature 1..9: note = RNA
  misc_feature 10..19: note = DNA
  misc_feature 20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = 2'-MOE guanosine-3'-phosphorothioate
  modified_base 10: mod_base = OTHER; note = thymidine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = Cytidine-3'-phosphorothioate
  modified_base 16..18: mod_base = OTHER; note = thymidine-3'-phosphorothioate
  modified_base 19: mod_base = OTHER; note = adenosine-3'-phosphorothioate
  modified_base 20: mod_base = OTHER; note = 2'-MOE uridine
agcttcttgtccagctttat

SEQ ID NO: 25 - DNA, 20 bp, synthetic construct
  misc_feature 1..10: note = RNA
  misc_feature 11..20: note = DNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE Cytidine-3'-phosphorothioate
  modified_base 4..5: mod_base = OTHER; note = 2'-MOE uridine-3'-phosphorothioate
  modified_base 6: mod_base = OTHER; note = 2'-MOE Cytidine-3'-phosphorothioate
  modified_base 7..8: mod_base = OTHER; note = 2'-MOE uridine-3'-phosphorothioate
  modified_base 9: mod_base = OTHER; note = 2'-MOE guanosine-3'-phosphorothioate
  modified_base 10: mod_base = OTHER; note = 2'-MOE uridine-3'-phosphorothioate
  modified_base 11..12: mod_base = OTHER; note = Cytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = Cytidine-3'-phosphorothioate
  modified_base 16..18: mod_base = OTHER; note = thymidine-3'-phosphorothioate
  modified_base 19: mod_base = OTHER; note = adenosine-3'-phosphorothioate
  modified_base 20: mod_base = OTHER; note = thymidine
agcttcttgtccagctttat

SEQ ID NO: 26 - skipped

SEQ ID NO: 27 - DNA, 10 bp, synthetic construct
  misc_feature 1: note = RNA
  misc_feature 2..9: note = DNA
  misc_feature 10: note = RNA
  source 1..10: mol_type = other DNA
  modified_base 1: mod_base = OTHER; note = 2'-MOE 5-methylCytidine-3'-phosphorothioate
  modified_base 2..3: mod_base = OTHER; note = thymidine-3'-phosphorothioate
  modified_base 4: mod_base = OTHER; note = guanosine-3'-phosphorothioate
  modified_base 5: mod_base = OTHER; note = thymidine-3'-phosphorothioate
  modified_base 6..7: mod_base = OTHER; note = 5-methylCytidine-3'-phosphorothioate
  modified_base 8: mod_base = OTHER; note = adenosine-3'-phosphorothioate
  modified_base 9: mod_base = OTHER; note = guanosine-3'-phosphorothioate
  modified_base 10: mod_base = OTHER; note = 2'-MOE 5-methylCytidine
cttgtccagc

SEQ ID NO: 28 - DNA, 12 bp, synthetic construct
  misc_feature 1..2: note = RNA
  misc_feature 3..10: note = DNA
  misc_feature 11..12: note = RNA
  source 1..12: mol_type = other DNA
  modified_base 1: mod_base = OTHER; note = 2'-MOE 5-methyluridine-3'-phosphorothioate
  modified_base 2: mod_base = OTHER; note = 2'-MOE 5-methylCytidine-3'-phosphorothioate
  modified_base 3..4: mod_base = OTHER; note = thymidine-3'-phosphorothioate
  modified_base 5: mod_base = OTHER; note = guanosine-3'-phosphorothioate
  modified_base 6: mod_base = OTHER; note = thymidine-3'-phosphorothioate
  modified_base 7..8: mod_base = OTHER; note = 5-methylCytidine-3'-phosphorothioate
  modified_base 9: mod_base = OTHER; note = adenosine-3'-phosphorothioate
  modified_base 10: mod_base = OTHER; note = guanosine-3'-phosphorothioate
  modified_base 11: mod_base = OTHER; note = 2'-MOE 5-methylCytidine-3'-phosphorothioate
  modified_base 12: mod_base = OTHER; note = 2'-MOE 5-methyluridine
tcttgtccagct

SEQ ID NO: 29 - DNA, 14 bp, synthetic construct
  misc_feature 1..2: note = RNA
  misc_feature 3..12: note = DNA
  misc_feature 13..14: note = RNA
  source 1..14: mol_type = other DNA
  modified_base 1..2: mod_base = OTHER; note = 2'-MOE 5-methyluridine-3'-phosphorothioate
  modified_base 3: mod_base = OTHER; note = 5-methylCytidine-3'-phosphorothioate
  modified_base 4..5: mod_base = OTHER; note = thymidine-3'-phosphorothioate
  modified_base 6: mod_base = OTHER; note = guanosine-3'-phosphorothioate
  modified_base 7: mod_base = OTHER; note = thymidine-3'-phosphorothioate
  modified_base 8..9: mod_base = OTHER; note = 5-methylCytidine-3'-phosphorothioate
  modified_base 10: mod_base = OTHER; note = adenosine-3'-phosphorothioate
  modified_base 11: mod_base = OTHER; note = guanosine-3'-phosphorothioate
  modified_base 12: mod_base = OTHER; note = 5-methylCytidine-3'-phosphorothioate
  modified_base 13: mod_base = OTHER; note = 2'-MOE 5-methyluridine-3'-phosphorothioate
  modified_base 14: mod_base = OTHER; note = 2'-MOE 5-methyluridine
ttcttgtccagctt

SEQ ID NO: 30 - DNA, 16 bp, synthetic construct
  misc_feature 1..3: note = RNA
  misc_feature 4..13: note = DNA
  misc_feature 14..16: note = RNA
  source 1..16: mol_type = other DNA
  modified_base 1: mod_base = OTHER; note = 2'-MOE 5-methylCytidine-3'-phosphorothioate
  modified_base 2..3: mod_base = OTHER; note = 2'-MOE 5-methyluridine-3'-phosphorothioate
  modified_base 4: mod_base = OTHER; note = 5-methylCytidine-3'-phosphorothioate
  modified_base 5..6: mod_base = OTHER; note = thymidine-3'-phosphorothioate
  modified_base 7: mod_base = OTHER; note = guanosine-3'-phosphorothioate
  modified_base 8: mod_base = OTHER; note = thymidine-3'-phosphorothioate
  modified_base 9..10: mod_base = OTHER; note = 5-methylCytidine-3'-phosphorothioate
  modified_base 11: mod_base = OTHER; note = adenosine-3'-phosphorothioate
  modified_base 12: mod_base = OTHER; note = guanosine-3'-phosphorothioate
  modified_base 13: mod_base = OTHER; note = 5-methylCytidine-3'-phosphorothioate
  modified_base 14..15: mod_base = OTHER; note = 2'-MOE 5-methyluridine-3'-phosphorothioate
  modified_base 16: mod_base = OTHER; note = 2'-MOE 5-methyluridine
cttcttgtccagcttt

SEQ ID NO: 31 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1: mod_base = OTHER; note = 2'-MOE adenosine-3'-phosphorothioate
  modified_base 2: mod_base = OTHER; note = 2'-MOE guanosine-3'-phosphorothioate
  modified_base 3: mod_base = OTHER; note = 2'-MOE 5-methylCytidine-3'-phosphorothioate
  modified_base 4..5: mod_base = OTHER; note = 2'-MOE 5-methyluridine-3'-phosphorothioate
  modified_base 6: mod_base = OTHER; note = 5-methylCytidine-3'-phosphorothioate
  modified_base 7..8: mod_base = OTHER; note = thymidine-3'-phosphorothioate
  modified_base 9: mod_base = OTHER; note = guanosine-3'-phosphorothioate
  modified_base 10: mod_base = OTHER; note = thymidine-3'-phosphorothioate
  modified_base 11..12: mod_base = OTHER; note = 5-methylCytidine-3'-phosphorothioate
  modified_base 13: mod_base = OTHER; note = adenosine-3'-phosphorothioate
  modified_base 14: mod_base = OTHER; note = guanosine-3'-phosphorothioate
  modified_base 15: mod_base = OTHER; note = 5-methylCytidine-3'-phosphorothioate
  modified_base 16..18: mod_base = OTHER; note = 2'-MOE 5-methyluridine-3'-phosphorothioate
  modified_base 19: mod_base = OTHER; note = 2'-MOE adenosine-3'-phosphorothioate
  modified_base 20: mod_base = OTHER; note = 2'-MOE 5-methyluridine
agcttcttgtccagctttat

SEQ ID NO: 32 - DNA, 24 bp, synthetic construct
  misc_feature 1..6: note = RNA
  misc_feature 7..18: note = DNA
  misc_feature 19..24: note = RNA
  source 1..24: mol_type = other DNA
  modified_base 1: mod_base = OTHER; note = 2'-MOE guanosine-3'-phosphorothioate
  modified_base 2: mod_base = OTHER; note = 2'-MOE 5-methylCytidine-3'-phosphorothioate
  modified_base 3: mod_base = OTHER; note = 2'-MOE adenosine-3'-phosphorothioate
  modified_base 4: mod_base = OTHER; note = 2'-MOE guanosine-3'-phosphorothioate
  modified_base 5: mod_base = OTHER; note = 2'-MOE 5-methylCytidine-3'-phosphorothioate
  modified_base 6: mod_base = OTHER; note = 2'-MOE 5-methyluridine-3'-phosphorothioate
  modified_base 7: mod_base = OTHER; note = thymidine-3'-phosphorothioate
  modified_base 8: mod_base = OTHER; note = 5-methylCytidine-3'-phosphorothioate
  modified_base 9..10: mod_base = OTHER; note = thymidine-3'-phosphorothioate
  modified_base 11: mod_base = OTHER; note = guanosine-3'-phosphorothioate
  modified_base 12: mod_base = OTHER; note = thymidine-3'-phosphorothioate
  modified_base 13..14: mod_base = OTHER; note = 5-methylCytidine-3'-phosphorothioate
  modified_base 15: mod_base = OTHER; note = adenosine-3'-phosphorothioate
  modified_base 16: mod_base = OTHER; note = guanosine-3'-phosphorothioate
  modified_base 17: mod_base = OTHER; note = 5-methylCytidine-3'-phosphorothioate
  modified_base 18: mod_base = OTHER; note = thymidine-3'-phosphorothioate
  modified_base 19..20: mod_base = OTHER; note = 2'-MOE 5-methyluridine-3'-phosphorothioate
  modified_base 21: mod_base = OTHER; note = 2'-MOE adenosine-3'-phosphorothioate
  modified_base 22..23: mod_base = OTHER; note = 2'-MOE 5-methyluridine-3'-phosphorothioate
  modified_base 24: mod_base = OTHER; note = 2'-MOE guanosine
gcagcttcttgtccagctttattg

SEQ ID NO: 33 - DNA, 28 bp, synthetic construct
  misc_feature 1..7: note = RNA
  misc_feature 8..21: note = DNA
  misc_feature 22..28: note = RNA
  source 1..28: mol_type = other DNA
  modified_base 1: mod_base = OTHER; note = 2'-MOE 5-methyluridine-3'-phosphorothioate
  modified_base 2: mod_base = OTHER; note = 2'-MOE adenosine-3'-phosphorothioate
  modified_base 3: mod_base = OTHER; note = 2'-MOE guanosine-3'-phosphorothioate
  modified_base 4: mod_base = OTHER; note = 2'-MOE 5-methylCytidine-3'-phosphorothioate
  modified_base 5: mod_base = OTHER; note = 2'-MOE adenosine-3'-phosphorothioate
  modified_base 6: mod_base = OTHER; note = 2'-MOE guanosine-3'-phosphorothioate
  modified_base 7: mod_base = OTHER; note = 2'-MOE 5-methylCytidine-3'-phosphorothioate
  modified_base 8..9: mod_base = OTHER; note = thymidine-3'-phosphorothioate
  modified_base 10: mod_base = OTHER; note = 5-methylCytidine-3'-phosphorothioate
  modified_base 11..12: mod_base = OTHER; note = thymidine-3'-phosphorothioate
  modified_base 13: mod_base = OTHER; note = guanosine-3'-phosphorothioate
  modified_base 14: mod_base = OTHER; note = thymidine-3'-phosphorothioate
  modified_base 15..16: mod_base = OTHER; note = 5-methylCytidine-3'-phosphorothioate
  modified_base 17: mod_base = OTHER; note = adenosine-3'-phosphorothioate
  modified_base 18: mod_base = OTHER; note = guanosine-3'-phosphorothioate
  modified_base 19: mod_base = OTHER; note = 5-methylCytidine-3'-phosphorothioate
  modified_base 20..21: mod_base = OTHER; note = thymidine-3'-phosphorothioate
  modified_base 22: mod_base = OTHER; note = 2'-MOE 5-methyluridine-3'-phosphorothioate
  modified_base 23: mod_base = OTHER; note = 2'-MOE adenosine-3'-phosphorothioate
  modified_base 24..25: mod_base = OTHER; note = 2'-MOE 5-methyluridine-3'-phosphorothioate
  modified_base 26..27: mod_base = OTHER; note = 2'-MOE guanosine-3'-phosphorothioate
  modified_base 28: mod_base = OTHER; note = 2'-MOE guanosine
tagcagcttcttgtccagctttattggg

SEQ ID NO: 34 - DNA, 32 bp, synthetic construct
  misc_feature 1..8: note = RNA
  misc_feature 9..24: note = DNA
  misc_feature 25..32: note = RNA
  source 1..32: mol_type = other DNA
  modified_base 1: mod_base = OTHER; note = 2'-MOE 5-methylCytidine-3'-phosphorothioate
  modified_base 2: mod_base = OTHER; note = 2'-MOE adenosine-3'-phosphorothioate
  modified_base 3: mod_base = OTHER; note = 2'-MOE 5-methyluridine-3'-phosphorothioate
  modified_base 4: mod_base = OTHER; note = 2'-MOE adenosine-3'-phosphorothioate
  modified_base 5: mod_base = OTHER; note = 2'-MOE guanosine-3'-phosphorothioate
  modified_base 6: mod_base = OTHER; note = 2'-MOE 5-methylCytidine-3'-phosphorothioate
  modified_base 7: mod_base = OTHER; note = 2'-MOE adenosine-3'-phosphorothioate
  modified_base 8: mod_base = OTHER; note = 2'-MOE guanosine-3'-phosphorothioate
  modified_base 9: mod_base = OTHER; note = 5-methylCytidine-3'-phosphorothioate
  modified_base 10..11: mod_base = OTHER; note = thymidine-3'-phosphorothioate
  modified_base 12: mod_base = OTHER; note = 5-methylCytidine-3'-phosphorothioate
  modified_base 13..14: mod_base = OTHER; note = thymidine-3'-phosphorothioate
  modified_base 15: mod_base = OTHER; note = guanosine-3'-phosphorothioate
  modified_base 16: mod_base = OTHER; note = thymidine-3'-phosphorothioate
  modified_base 17..18: mod_base = OTHER; note = 5-methylCytidine-3'-phosphorothioate
  modified_base 19: mod_base = OTHER; note = adenosine-3'-phosphorothioate
  modified_base 20: mod_base = OTHER; note = guanosine-3'-phosphorothioate
  modified_base 21: mod_base = OTHER; note = 5-methylCytidine-3'-phosphorothioate
  modified_base 22..24: mod_base = OTHER; note = thymidine-3'-phosphorothioate
  modified_base 25: mod_base = OTHER; note = 2'-MOE adenosine-3'-phosphorothioate
  modified_base 26..27: mod_base = OTHER; note = 2'-MOE 5-methyluridine-3'-phosphorothioate
  modified_base 28..30: mod_base = OTHER; note = 2'-MOE guanosine-3'-phosphorothioate
  modified_base 31: mod_base = OTHER; note = 2'-MOE adenosine-3'-phosphorothioate
  modified_base 32: mod_base = OTHER; note = 2'-MOE guanosine
catagcagcttcttgtccagctttattgggag

SEQ ID NO: 35 - DNA, 36 bp, synthetic construct
  misc_feature 1..8: note = RNA
  misc_feature 9..26: note = DNA
  misc_feature 27..36: note = RNA
  source 1..36: mol_type = other DNA
  modified_base 1: mod_base = OTHER; note = 2'-MOE 5-methyluridine-3'-phosphorothioate
  modified_base 2: mod_base = OTHER; note = 2'-MOE 5-methylCytidine-3'-phosphorothioate
  modified_base 3: mod_base = OTHER; note = 2'-MOE adenosine-3'-phosphorothioate
  modified_base 4: mod_base = OTHER; note = 2'-MOE 5-methyluridine-3'-phosphorothioate
  modified_base 5: mod_base = OTHER; note = 2'-MOE adenosine-3'-phosphorothioate
  modified_base 6: mod_base = OTHER; note = 2'-MOE guanosine-3'-phosphorothioate
  modified_base 7: mod_base = OTHER; note = 2'-MOE 5-methylCytidine-3'-phosphorothioate
  modified_base 8: mod_base = OTHER; note = 2'-MOE adenosine-3'-phosphorothioate
  modified_base 9: mod_base = OTHER; note = guanosine-3'-phosphorothioate
  modified_base 10: mod_base = OTHER; note = 5-methylCytidine-3'-phosphorothioate
  modified_base 11..12: mod_base = OTHER; note = thymidine-3'-phosphorothioate
  modified_base 13: mod_base = OTHER; note = 5-methylCytidine-3'-phosphorothioate
  modified_base 14..15: mod_base = OTHER; note = thymidine-3'-phosphorothioate
  modified_base 16: mod_base = OTHER; note = guanosine-3'-phosphorothioate
  modified_base 17: mod_base = OTHER; note = thymidine-3'-phosphorothioate
  modified_base 18..19: mod_base = OTHER; note = 5-methylCytidine-3'-phosphorothioate
  modified_base 20: mod_base = OTHER; note = adenosine-3'-phosphorothioate
  modified_base 21: mod_base = OTHER; note = guanosine-3'-phosphorothioate
  modified_base 22: mod_base = OTHER; note = 5-methylCytidine-3'-phosphorothioate
  modified_base 23..25: mod_base = OTHER; note = thymidine-3'-phosphorothioate
  modified_base 26: mod_base = OTHER; note = adenosine-3'-phosphorothioate
  modified_base 27..28: mod_base = OTHER; note = 2'-MOE 5-methyluridine-3'-phosphorothioate
  modified_base 29..31: mod_base = OTHER; note = 2'-MOE guanosine-3'-phosphorothioate
  modified_base 32: mod_base = OTHER; note = 2'-MOE adenosine-3'-phosphorothioate
  modified_base 33..34: mod_base = OTHER; note = 2'-MOE guanosine-3'-phosphorothioate
  modified_base 35: mod_base = OTHER; note = 2'-MOE 5-methylCytidine-3'-phosphorothioate
  modified_base 36: mod_base = OTHER; note = 2'-MOE 5-methylCytidine
tcatagcagcttcttgtccagctttattgggaggcc

SEQ ID NO: 36 - RNA, 20 nt, Homo sapiens
  source 1..20: mol_type = mRNA
ataaagctggacaagaagct

SEQ ID NO: 37 - RNA, 20 nt, synthetic construct
  source 1..20: mol_type = other RNA
agcttcttgtccagctttat